(19) United States
(12) Patent Application Publication
(10) Pub. No.: US 2024/0400521 A1
(43) Pub. Date: Dec. 5, 2024

(54) QUINAZOLINE COMPOUND, COMPOSITION, AND APPLICATION THEREOF

(71) Applicant: BEIJING SCITECH-MQ PHARMACEUTICALS LIMITED, Beijing (CN)

(72) Inventors: Qiang ZHANG, Beijing (CN); Leifu YANG, Beijing (CN); Yueming SUN, Beijing (CN)

(73) Assignee: BEIJING SCITECH-MQ PHARMACEUTICALS LIMITED, Beijing (CN)

(21) Appl. No.: 18/697,150

(22) PCT Filed: Sep. 29, 2022
(86) PCT No.: PCT/CN2022/122577

§ 371 Date: Mar. 29, 2024

(30) Foreign Application Priority Data
Sep. 30, 2021 (CN) 202111158182.1

(51) Int. Cl. C07D 239/94; A61K 31/517; A61P 35/04

(52) U.S. Cl. CPC C07D 239/94; A61K 31/517; A61P 35/04

(57) ABSTRACT

Provided are a quinazoline compound, a composition, and an application thereof, in particular, a compound represented by formula (I), a stereoisomer thereof, a pharmaceutically acceptable salt thereof, a composition thereof, and an application thereof in the preparation for a drug that serves as a tyrosine kinase inhibitor. The compound represented by formula (I) has good inhibitory activity against EGFR and HER2 kinases.

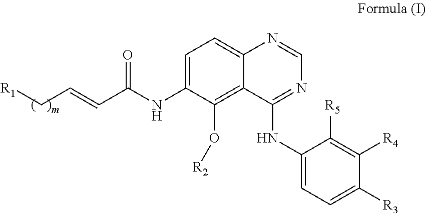

### CROSS REFERENCE TO RELATED APPLICATION

This application is a National Stage filing under 35 U.S.C. 371 of International PCT Application No. PCT/CN2022/122577, filed on Sep. 29, 2022, which claims the priority of CN202111158182.1, filed on Sep. 30, 2021. The Chinese Patent Application No. CN202111158182.1 is incorporated herein by reference as part of the disclosure of the present application.

### FIELD OF THE INVENTION

The present disclosure belongs to the field of pharmaceutical technology and relates to quinazoline-based compounds, compositions and applications thereof.

### BACKGROUND OF THE INVENTION

Epidermal growth factor receptor (ErbB) tyrosine kinases can regulate cell proliferation, migration, differentiation, apoptosis, and cell movement through multiple pathways. ErbB family members, as well as some of their ligands, are commonly overexpressed, amplified, or mutated in many forms of malignancies, making them important cancer therapeutic targets. This family of protein kinases includes ErbB1/EGFR/HER1, ErbB2/HER2, ErbB3/HER3, and ErbB4/HER4, and several kinase inhibitors for treating non-small cell lung cancer and breast cancer have been successfully developed based on EGFR and HER2. (Dienstmann R., et. al., (2001) Personalizing Therapy with Targeted Agents in Non-Small Cell Lung Cancer. ONCOTARGET. 2 (3), 165.; Mitri Z., et. al. (2012) The HER2 Receptor in Breast Cancer: Pathophysiology, Clinical Use, and New Advances in Therapy., Chemotherapy Research & Practice., Volum 2012 (23), 743193.).

EGFR is widely expressed and plays an important role in growth and development and normal physiological functions. Moreover, EGFR and the signaling pathways it mediates also play an important role in the occurrence and development of tumors. However, the expression of EGFR is very unstable, and gene amplification and rearrangement often occur, which changes the antigen phenotype on the surface of tumor cells, wherein epidermal growth factor receptor variant III (EGFRvIII) is the most common.

EGFRvIII is a type of epidermal growth factor receptor (EGFR) mutant discovered in recent years that is expressed on the surface of only tumor cells but not normal tissue cells. Abnormal expression of EGFR is associated with the occurrence of many malignant tumors, including glioma, lung small cell carcinoma, breast cancer, bladder cancer, ovarian cancer, etc.

Compared with the complete structure of EGFR, exons 2-7 encoding the extracellular ligand binding region of EGFRvIII are deleted, resulting in a deletion of 801 base pairs, allowing exons 1 and 8 to be connected and a new glycine is generated at the binding site, resulting in the deletion of amino acids 6 to 273, thus losing the ability to bind to the ligand EGF. In the absence of ligand binding, EGFRvIII causes unregulated structural activation of tyrosine kinases through dimerization and autophosphorylation, inducing downstream signaling and stimulating tumor cell proliferation.

Existing studies have shown that EGFRvIII can affect the occurrence and development of tumors by regulating multiple signaling pathways, including Ras/Raf/MEK/ERK, PI3/AKT/mTOR, JAK/STAT and PLC/PKC, etc. EGFRvIII-positive tumor cells have significantly increased tumorigenicity, mainly by inhibiting apoptosis, promoting tumor angiogenesis, increasing invasiveness and migration, etc., leading to uncontrollable spontaneous proliferation and metastasis of tumor cells. In addition, EGFRvIII plays an escape-like function during tumor radiotherapy and chemotherapy.

Glioma is a common malignant tumor with high invasiveness, and glioblastoma (GBM) is the most malignant type. The effects of radiotherapy and chemotherapy against glioblastoma are not ideal, and recurrences often occur after surgery. Studies in China and foreign countries have found that 40% to 60% of GBM significantly express EGFR, and the mutant form is mainly EGFRvIII. EGFRvIII establishes a signaling pathway regulatory network through receptor-independent autophosphorylation and tyrosine kinase activity, and plays an important role in regulating the growth, metastasis and angiogenesis of GBM.

In recent years, studies have found that EGFRvIII molecularly targeted treatments have shown good anti-tumor effects in both in vitro cell culture and in vivo animal models. Therefore, the development of new molecularly targeted therapeutic drugs targeting EGFRvIII will provide more effective and cost-effective treatment options for tumor patients, especially glioma patients, and there is a huge unmet clinical need.

Medicaments targeting EGFRvIII to treat glioma not only need to be able to effectively penetrate the blood-brain barrier, but also need to be able to effectively inhibit EGFRvIII. There are currently no reports of compounds that can both penetrate the blood-brain barrier and inhibit EGFRvIII. Therefore, research on EGFRvIII-driven gliomas has important clinical value. In addition, the vast majority of marketed EGFR and HER2 kinase inhibitors are unable to penetrate the blood-brain barrier, and patients with EGFR-driven lung cancer and HER2-driven breast cancer generally have poor prognosis and a higher risk of brain metastasis. There are currently no effective medicaments approved for the treatment of brain metastases, so there is an urgent need to develop an EGFR inhibitor and/or HER2 inhibitor that can penetrate the blood-brain barrier.

### SUMMARY OF THE INVENTION

One aspect of the present disclosure provides a compound represented by formula (I), a stereoisomer thereof, and a pharmaceutically acceptable salt thereof, wherein

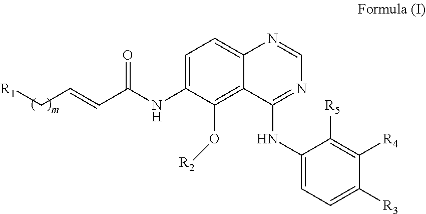

in formula (I), m is 0, 1 or 2;
R₁ is hydrogen, 4- to 7-membered heteroalicyclyl or —NR^{a}R^{b},
R^{a} and R^{b} are each independently hydrogen, C₁-C₆ alkyl, C₃-C₆ cycloalkyl, hydroxy-substituted C₁-C₆ alkyl, C₁-C₃ alkoxy-substituted C₁-C₆ alkyl, or C₃-C₆ cycloalkyl-substituted C₁-C₆ alkyl,
the 4- to 7-membered heteroalicyclyl group is a heteroalicyclyl group containing 1-2 heteroatoms selected from N, O and S, wherein the heteroalicyclyl group is unsubstituted or substituted with one or two of C₁-C₃ alkyl, C₁-C₄ acyl, hydroxyl, cyano, aminoacyl, mono- or di-C₁-C₃ alkyl-substituted aminoacyl, C₁-C₃ alkylsulfonyl, C₁-C₃ alkyl sulfoxide group, and oxo (═O);
R₂ is C₁-C₆ alkyl, which is unsubstituted or substituted with 1 to 3 substituents selected from halogen, cyano, C₁-C₃ alkyl, C₁-C₃ alkoxy, C₁-C₃ alkylthio, hydroxyl, C₃-C₄ cycloalkyl and C₁-C₃ alkylsulfonyl;
R₃, R₄ and R₅ are each independently hydrogen, halogen, C₁-C₃ alkyl, C₁-C₃ alkoxy, or C₃-C₄ cycloalkyl, and at least one of R₃, R₄ and R₅ is halogen.

According to an alternative embodiment, m is 0 or 1,
R₁ is 4- to 7-membered heteroalicyclyl or —NR^{a}R^{b},
R^{a} and R^{b} are each independently hydrogen, C₁-C₃ alkyl, C₃-C₆ cycloalkyl, hydroxy-substituted C₁-C₃ alkyl, C₁-C₃ alkoxy-substituted C₁-C₃ alkyl;
the 4- to 7-membered heteroalicyclyl group is pyrrolidinyl, piperidinyl, piperazinyl, morpholinyl, tetrahydrofuranyl, tetrahydropyranyl, or thiomorpholinyl, and the above groups are unsubstituted or substituted with one or two of methyl, ethyl, propyl, isopropyl, aldehyde group, acetyl, propionyl, hydroxy, cyano, aminoacyl, methyl sulfonyl, ethyl sulfonyl, propyl sulfonyl, isopropyl sulfonyl, methyl sulfoxide group, ethyl sulfoxide group, propyl sulfoxide group, isopropyl sulfoxide group, and oxo (═O).

R₁ is 1-methylpyrrolidin-2-yl, 1-ethylpyrrolidin-2-yl, 1-propylpyrrolidin-2-yl, 1-isopropylpyrrolidin-2-yl, pyrrolidin-1-yl, piperidin-1-yl, 1-methylpiperazin-4-yl, 1-ethylpiperazin-4-yl, morpholinyl, tetrahydrofuran-2-yl, tetrahydrofuran-3-yl, tetrahydropyran-2-yl, tetrahydropyran-3-yl, tetrahydropyran-4-yl, thiomorpholinyl, dimethylamino, diethylamino, dipropylamino, diisopropylamino, methylethylamino, methylpropylamino, methylamino, ethylamino, propylamino, isopropylamino, cyclopropylamino, cyclobutylamino, methylisopropylamino, N-methyl-N-cyclopropylamino, N-methyl-N-cyclobutylamino or ethylpropylamino.

According to an alternative embodiment, R₂ is C₁-C₄ alkyl, which is unsubstituted or substituted with 1 to 3 substituents selected from fluorine, chlorine, cyano, methyl, ethyl, propyl, isopropyl, methoxy, ethoxy, propoxy, isopropoxy, methylthio, ethylthio, propylthio, isopropylthio, hydroxyl, cyclopropyl, cyclobutyl, methylsulfonyl, ethylsulfonyl, propylsulfonyl and isopropylsulfonyl.

Yet alternatively, R₂ is methyl, ethyl, propyl, isopropyl, hydroxyethyl, hydroxypropyl, trifluoromethyl, fluoroethyl, fluoropropyl, 2,2,2-trifluoroethyl, 2,2-difluoroethyl, 3,3,3-trifluoropropyl, methoxyethyl, methoxypropyl, ethoxyethyl, ethoxypropyl, methylthioethyl, methylthiopropyl, ethylthioethyl, ethylthiopropyl, 2-hydroxy-2-methylpropyl, 3-hydroxy-3-methylbutyl, methylsulfonylpropyl, methylsulfonylethyl, ethylsulfonylethyl, ethylsulfonylpropyl, isopropylsulfonylethyl, or isopropylsulfonylpropyl.

According to an alternative embodiment, R₃, R₄ and R₅ are each independently hydrogen, fluorine, chlorine, or bromine, and at least one of R₃, R₄, and R₅ is fluorine, chlorine, or bromine.

Yet alternatively, R₃ and R₅ are each independently hydrogen, fluorine, or chlorine, and R₄ is chlorine.

Still yet alternatively, R₃ is hydrogen, fluorine, or chlorine, R₄ is chlorine, and R₅ is fluorine.

Typical compounds involved in this application are as follows:

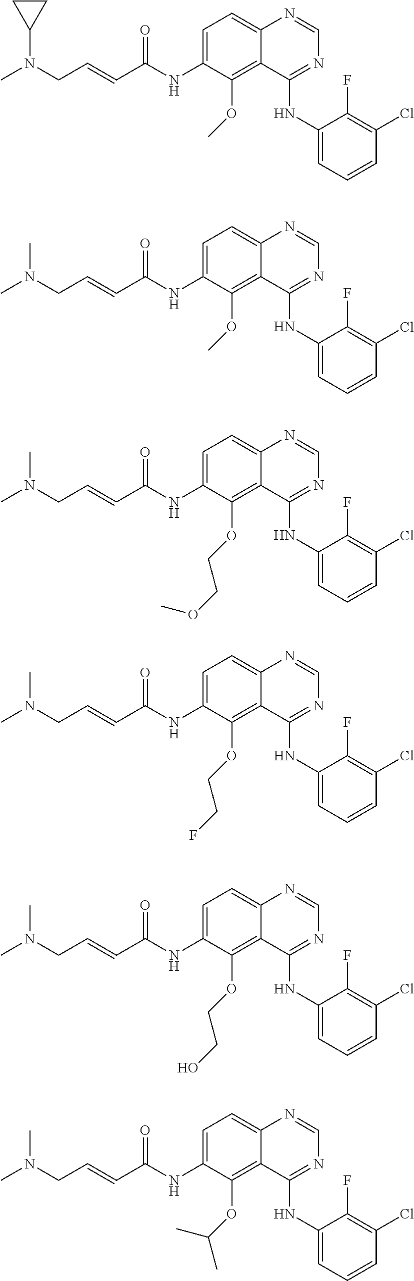 [SMILES: COc1c(NC(=O)/C=C/CN(C)C2CC2)ccc2ncnc(Nc3cccc(Cl)c3F)c12 ; COc1c(NC(=O)/C=C/CN(C)C)ccc2ncnc(Nc3cccc(Cl)c3F)c12 ; COCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCF ; CN(C)C/C=C/C(=O)Nc1ccc2ncnc(Nc3cccc(Cl)c3F)c2c1OCCO ; CC(C)Oc1c(NC(=O)/C=C/CN(C)C)ccc2ncnc(Nc3cccc(Cl)c3F)c12]

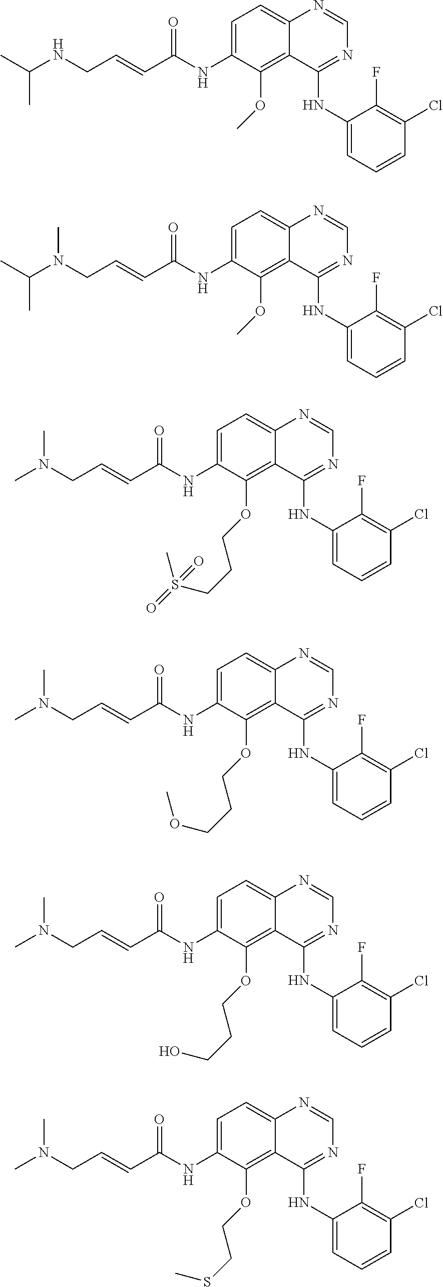 [SMILES: COc1c(NC(=O)/C=C/CNC(C)C)ccc2ncnc(Nc3cccc(Cl)c3F)c12 ; COc1c(NC(=O)/C=C/CN(C)C(C)C)ccc2ncnc(Nc3cccc(Cl)c3F)c12 ; CN(C)C/C=C/C(=O)Nc1ccc2ncnc(Nc3cccc(Cl)c3F)c2c1OCCCS(C)(=O)=O ; COCCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCCO ; CSCCOc1c(NC(=O)/C=C/CN(C)C)ccc2ncnc(Nc3cccc(Cl)c3F)c12]

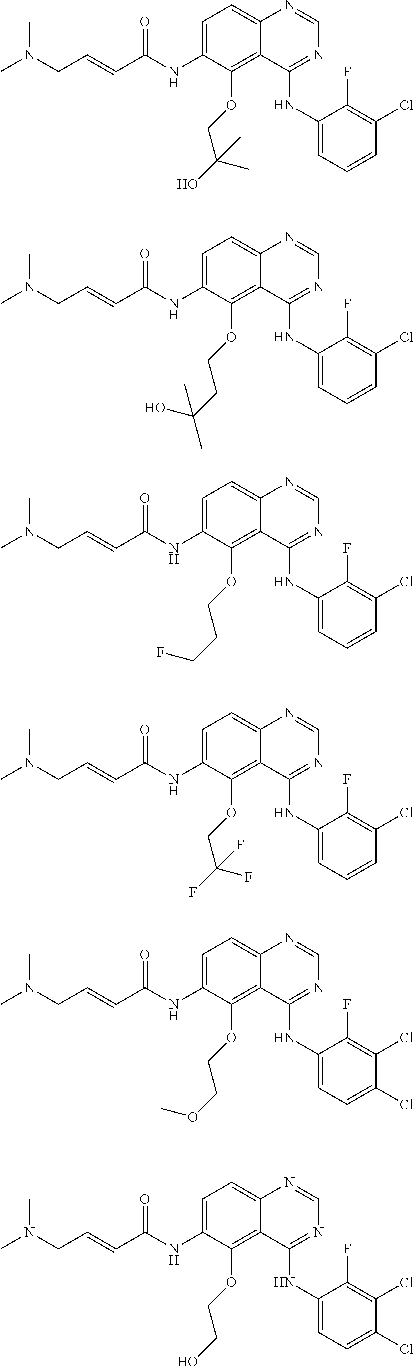

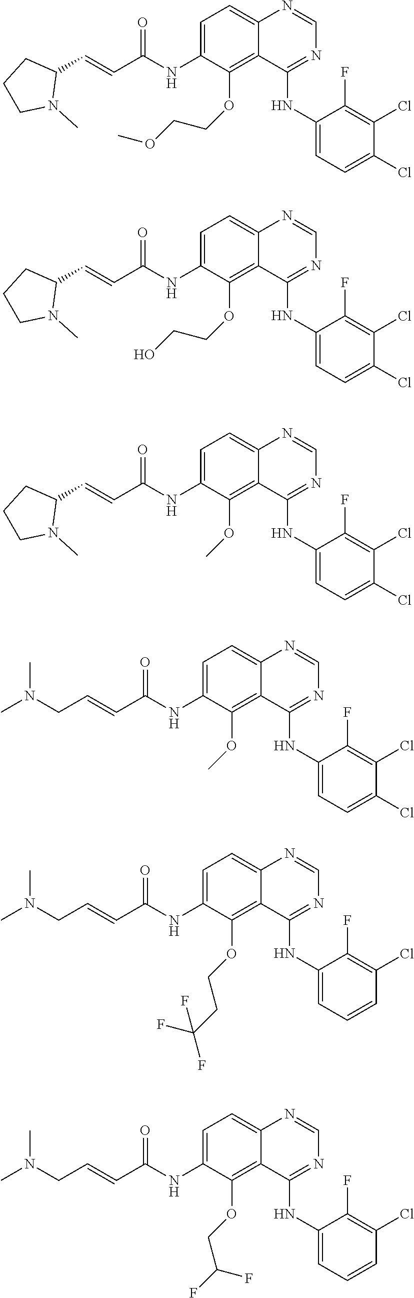

Another aspect of the present disclosure provides a pharmaceutical composition comprising a compound described herein, a pharmaceutically acceptable salt, stereoisomer, solvate, or prodrug thereof, and one or more pharmaceutically acceptable carrier(s) or excipient(s).

The pharmaceutical composition of the present application may also contain one or more other therapeutic agent(s).

The present disclosure also relates to a method for treating EGFR or HER2 kinase-mediated diseases or conditions, which includes administering to a patient in need (human or other mammal, especially human) a therapeutically effective amount of the compounds or salts thereof described in the present application, wherein the diseases or conditions mediated by EGFR or HER2 kinase include those mentioned above.

### DETAILED DESCRIPTION OF THE INVENTION

Unless otherwise stated, the following terms used in this application (including the specification and claims) have the definitions given below. In this application, the use of “or” or “and” means “and/or” unless stated otherwise. In addition, the use of the term “comprising” and other forms such as “including”, “containing” and “having” is not limiting. The chapter headings used herein are for organizational purposes only and should not be interpreted as limitations on the topics described.

Unless otherwise specified, an alkyl group refers to a saturated linear and branched hydrocarbon group having a specified number of carbon atoms, and the term C₁-C₆ alkyl refers to an alkyl moiety containing from 1 to 6 carbon atoms. Similarly, C₁-C₃ alkyl refers to an alkyl moiety containing from 1 to 3 carbon atoms. For example, C₁-C₆ alkyl includes methyl, ethyl, propyl, isopropyl, n-butyl, isobutyl, sec-butyl, tert-butyl, n-pentyl, 3-(2-methyl)butyl, 2-pentyl, 2-methylbutyl, neopentyl, n-hexyl, 2-hexyl and 2-methylpentyl, etc.

When a substituent term such as “alkyl” is used in combination with other substituent term, such as in terms “C₁-C₃ alkoxy C₁-C₆ alkylthio” or “hydroxy-substituted C₁-C₆ alkyl”, the linking substituent terms (e.g., alkyl or alkylthio) are intended to encompass a divalent moiety, wherein the point of attachment is through the linking substituent. Examples of “C₁-C₃ alkoxy C₁-C₆ alkylthio” include, but are not limited to, methoxymethylthio, methoxyethylthio and ethoxypropylthio, etc. Examples of “hydroxy-substituted C₁-C₆ alkyl” include, but are not limited to, hydroxymethyl, hydroxyethyl, and hydroxyisopropyl, etc.

An alkoxy group is an alkyl-O— group formed by a linear or branched alkyl group described previously and —O—, e.g., methoxy, ethoxy, and the like. Similarly, an alkylthio group is an alkyl-S— group formed by a linear or branched alkyl group as described previously and —S—, e.g., methylthio, ethylthio, and the like.

Alkenyl and alkynyl groups include linear or branched alkenyl or alkynyl groups, and the term C₂-C₆ alkenyl or C₂-C₆ alkynyl refers to linear or branched hydrocarbon groups having at least one alkenyl or alkynyl group.

The term “haloalkyl”, such as “C₁-C₆haloalkyl”, refers to a group having one or more halogen atoms, which may be the same or different, on one or more carbon atoms of an alkyl moiety comprising 1 to 6 carbon atoms. Examples of “C₁-C₆ haloalkyl” may include, but are not limited to, —CF₃ (trifluoromethyl), —CCl₃ (trichloromethyl), 1,1-difluoroethyl, 2,2,2-trifluoroethyl and hexafluoroisopropyl, etc. Similarly, the term “C₁-C₆haloalkoxy” refers to a haloalkyl-O— group formed by the C₁-C₆haloalkyl and —O—, which can be, for example, trifluoromethoxy, trichloromethoxy, etc.

The term “C₁-C₄ acyl” includes formyl (aldehyde group) (—CHO), acetyl (CH₃CO—), propionyl (C₂H₅CO—) and the like. The term “aminoacyl” refers to NH₂CO—.

The term “cycloalkyl” refers to a non-aromatic, saturated, cyclic hydrocarbon group containing a specified number of carbon atoms. For example, the term “(C₃-C₆)cycloalkyl” refers to a non-aromatic cyclic hydrocarbon ring having 3-6 ring carbon atoms. Exemplary “(C₃-C₆)cycloalkyl” includes cyclopropyl, cyclobutyl, cyclopentyl, and cyclohexyl.

The term “aryl” refers to a group or moiety comprising an aromatic monocyclic or bicyclic hydrocarbon radical, which contains from 6 to 12 carbon ring atoms and has at least one aromatic ring. Examples of “aryl” are phenyl, naphthyl, indenyl and dihydroindenyl (indanyl). Generally, in the compounds of the present disclosure, the aryl is phenyl.

Unless otherwise specified, the term “heteroalicyclyl” as used herein refers to an unsubstituted or substituted stable 4- to 7-membered non-aromatic monocyclic saturated ring system consisting of carbon atoms and 1 to 3 heteroatoms selected from N, O, and S, wherein the N or S heteroatom can be randomly oxidized, and the N heteroatom can also be randomly quaternized. Examples of such heterocycles include, but are not limited to, azetidinyl, oxetanyl, thietanyl, pyrrolidinyl, pyrrolinyl, pyrazolidinyl, pyrazolinyl, imidazolidinyl, imidazolinyl, oxazolinyl, thiazolinyl, tetrahydrofuranyl, dihydrofuranyl, tetrahydrothienyl, 1,3-dioxolanyl, piperidinyl, piperazinyl, tetrahydropyranyl, dihydropyranyl, tetrahydrothiopyranyl, 1,3-dioxanyl, 1,4-dioxanyl, 1,3-oxathiolanyl, 1,3-oxathianyl, 1,3-dithianyl, 1,4-oxathiolanyl, 1,4-oxathianyl, 1,4-dithianyl, morpholinyl, and thiomorpholinyl.

The term “carbonyl” refers to a —C(O)— group. The terms “halogen” and “halo” refer to chlorine, fluorine, bromine or iodine substituent. “Oxo” refers to an oxygen moiety with double bond; for example, “oxo” may be directly attached to a carbon atom to form a carbonyl moiety (C═O). “Hydroxy” is intended to refer to a radical —OH. The term “cyano” as used herein refers to a group —CN.

The term “each independently” means that where more than one substituent is selected from a number of possible substituents, those substituents may be the same or different.

It is clear that the compound of formula I, or isomer, crystalline form or prodrug thereof, and pharmaceutically acceptable salt thereof, may exist in a solvated or non-solvated form. For example, the solvated form can be a water-soluble form. The present disclosure includes all the solvated and non-solvated forms.

In this disclosure, the term “isomer” refers to different compounds having the same molecular formula, and may include various isomeric forms such as stereoisomers and tautomers. “Stereoisomers” are isomers that differ only in the arrangement of their atoms in space. Some compounds described herein contain one or more asymmetric centers and thus can give rise to enantiomers, diastereomers, and other stereoisomeric forms which can be defined as (R)- or (S)-based on absolute stereochemistry. The chemical entities, pharmaceutical compositions, and methods disclosed herein are intended to include all of these possible isomers, including racemic mixtures, optically pure forms, and intervenient mixtures between them. Optically active (R)- and (S)-isomers can be prepared using chiral synthons or chiral reagents or resolved using conventional techniques. The optical activity of a compound can be analyzed by any suitable method, including but not limited to chiral chromatography and polarimetry, and the degree of dominance of one stereoisomer over other isomers can be determined.

Individual stereoisomers (or isomers-enriched mixtures) of a compound of this disclosure may be resolved using methods known to those skilled in the art. For example, such resolution may be carried out by: (1) formation of diastereoisomeric salts, complexes or other derivatives; (2) selective reaction with a stereoisomer-specific reagent, for example by enzymatic oxidation or reduction; or (3) gas-liquid or liquid chromatography in a chiral environment, for example, on a chiral support such as silica bound with chiral ligands or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

When a compound described herein contains an olefinic double bond, it means that the compound includes various cis- or trans-isomers, unless otherwise stated.

“Tautomers” are structurally different isomers that are interconvertible to each other through tautomerization. “Tautomerization” is a form of isomerization and includes a prototropic change or proton transfer tautomerization, which can be considered as a subset of acid-base chemistry. “prototropic change tautomerization” or “proton transfer tautomerization” involves the migration of a proton accompanied by a bond-level transformation, which is often exchange of a single bond with an adjacent double bond. When tautomerization is possible (for example, in solution), tautomers can reach chemical equilibrium. An example of tautomerization is keto-enol tautomerization.

The compound of the present disclosure as an active ingredient, and the method of preparing the same, are both included in the present disclosure. Moreover, the crystalline form of some of the compounds may exist as polymorphs, and such forms may also be included in the present disclosure. Additionally, some of the compounds may form solvates with water (i.e., hydrates) or common organic solvents, and such solvates are also included within the scope of the disclosure.

The compounds of the disclosure may be used in the free form for treatment or, when appropriate, in the form of a pharmaceutically acceptable salt or other derivative for treatment. As used herein, the term “pharmaceutically acceptable salt” refers to organic and inorganic salts of the compounds of the present disclosure which are suitable for use in human and lower animals without undue toxicity, irritation, allergic response, etc., and have reasonable benefit/risk ratio. Pharmaceutically acceptable salts of amines, carboxylic acids, phosphonates, and other types of compounds are well known in the art. The salt can be formed by reacting a compound of the disclosure with a suitable free base or acid, including, but not limited to, inorganic acids such as hydrochloric acid, hydrobromic acid, phosphoric acid, sulfuric acid, perchloric acid or organic acids such as acetic acid, oxalic acid, maleic acid, tartaric acid, citric acid, succinic acid, malonic acid. Or the salts may be obtained by methods well known in the art, such as ion exchange. Other pharmaceutically acceptable salts include adipate, alginate, ascorbate, aspartate, besylate, benzoate, bisulfate, borate, butyrate, camphorate, camphorsulfonate, citrate, digluconate, lauryl sulfate, ethanesulfonate, formate, fumarate, glucoheptonate, glycerol phosphate, glyconate, hemisulfate, hexanoate, hydroiodide, 2-hydroxyethanesulfonate, lactobionate, lactate, laurate, lauryl sulfate, malate, maleate, mesylate, 2-naphthalenesulfonate, nicotinate, nitrate, oleate, palmitate, pamoate, pectate, persulphate, per-3-phenylpropionate, phosphate, picrate, propionate, stearate, sulfate, thiocyanate, p-toluenesulfonate, undecanoate, and the like. Representative alkali or alkaline earth metal salts include salts of sodium, lithium, potassium, calcium, magnesium, and the like. Other pharmaceutically acceptable salts include suitable non-toxic salts of ammonium, quaternary ammonium, and amine cations formed from halides, hydroxides, carboxylates, sulfates, phosphates, nitrates, lower alkyl sulfonates and aryl sulfonates.

Further, the term “prodrug” as used herein means that a compound can be converted into the compound of the present disclosure in vivo. Such transformation is affected by hydrolysis of the prodrug in the blood or enzymatic conversion to the parent compound in the blood or tissue.

Pharmaceutical compositions of this disclosure comprise the compound described herein or a pharmaceutically acceptable salt thereof; an additional active agent selected from a kinase inhibitory agent (small molecule, polypeptide, antibody, etc.), an immunosuppressant, an anticancer agent, an anti-viral agent, anti-inflammatory agent, antifungal agent, antibiotic, and an anti-vascular hyper proliferation compound; and any pharmaceutically acceptable carrier, adjuvant or excipient.

The compounds of the present disclosure may be used alone or in combination with one or more of other compounds of the present disclosure or with one or more of other agents. When administered in combination, the therapeutic agents can be formulated for simultaneous or sequential administration at different times, or the therapeutic agents can be administered as a single composition. By “combination therapy”, it refers to the use of a compound of the present disclosure in combination with another agent, in a manner whereby each agent is co-administered simultaneously or administered sequentially, in either case, for the purpose of achieving the optimal effect of drugs. Co-administration includes dosage form for simultaneous delivery, as well as separate dosage forms for each compound. Thus, administration of the compounds of the disclosure can be combined with other therapies known in the art, for example, radiation therapy or cytostatic agents, cytotoxic agents, other anticancer agents, and the like as used in the treatment of cancer, in order to improve the symptoms of cancer. The administration sequence is not limited in the present disclosure. The compounds of the present disclosure may be administered before, simultaneously, or after other anticancer or cytotoxic agents.

To prepare the pharmaceutical ingredient of the present disclosure, one or more compounds of Formula (I) or salts thereof as an active ingredient can be intimately mixed with a pharmaceutical carrier, which is carried out according to conventional pharmaceutical formulation techniques. The carrier can be used in a wide variety of forms depending on the form of preparation which is designed for different modes of administration (for example, oral or parenteral administration). Suitable pharmaceutically acceptable carriers are well known in the art. A description of some of these pharmaceutically acceptable carriers can be found in the Handbook of Pharmaceutical Excipients, published jointly by the American Pharmaceutical Association and the Pharmaceutical Society of Great Britain.

The pharmaceutical composition of the present disclosure may have the following forms, for example, those suitable for oral administration, such as tablets, capsules, pills, powders, sustained release forms, solutions or suspensions; those for parenteral injections such as clear solutions, suspensions, emulsion; or those for topical application such as ointments, creams; or as a suppository for rectal administration. The pharmaceutical ingredients may also be presented in unit dosage form for single administration in a precise dosage. The pharmaceutical ingredient will include a conventional pharmaceutical carrier or excipient and a compound as an active ingredient prepared according to the present disclosure, and may also include other medical or pharmaceutical preparations, carriers, adjuvants, and the like.

Therapeutic compounds can also be administered to mammals other than humans. The drug dosage for a mammal will depend on the species of the animal and its disease condition or its disordered condition. The therapeutic compound can be administered to the animal in the form of a capsule, a bolus, a tablet or liquid. The therapeutic compound can also be introduced into the animal by injection or infusion. These drug forms are prepared in a traditional manner complying with standard veterinary practice. As an alternative, the pharmaceutical synthetic drugs can be fed to the animal in a mixture with the animal feed, so that the concentrated feed additive or premix can be prepared by mixing ordinary animal feed.

It is a further object of the present disclosure to provide a method for treating cancer in a subject in need thereof, comprising administering to the subject a therapeutically effective amount of a composition containing the compound of the present disclosure.

The present disclosure also includes the use of the compound of the present disclosure or a pharmaceutically acceptable derivative thereof, especially in the manufacture of a medicament for treating cancers and autoimmune diseases related to tyrosine kinase EGFR or HER2. The present disclosure also includes a medicament for cancers (including non-solid tumors, solid tumors, primary or metastatic cancers, as noted and included elsewhere herein, wherein the cancer may be resistant or refractory to one or more other treatments) and other disease (including but not limited to fundus disease, psoriasis, atherosclerosis, pulmonary fibrosis, liver fibrosis, bone marrow fibrosis, etc.). The cancer includes, but is not limited to any one of: non-small cell lung cancer, small cell lung cancer, breast cancer, pancreatic cancer, glioma, glioblastoma, ovarian cancer, cervical cancer, colorectal cancer, melanoma, endometrial cancer, prostate cancer, bladder cancer, leukemia, stomach cancer, liver cancer, gastrointestinal stromal tumor, thyroid cancer, chronic myelogenous leukemia, acute myeloid leukemia, non-Hodgkin lymphoma, nasopharyngeal cancer, esophageal cancer, brain tumors, B-cell and T-cell lymphoma, lymphoma, multiple myeloma, biliary carcinosarcoma, cholangiocarcinoma.

### EXAMPLES

The present disclosure also provides methods for preparing the corresponding compounds. Various synthetic methods can be used to prepare the compounds described herein, including the following methods. The compounds of the present disclosure, or pharmaceutically acceptable salts, isomers or hydrates thereof can be synthesized using the methods described below, synthetic methods known in the art of organic chemistry synthesis, or variants of these methods understood by those skilled in the art. Alternative methods include, but are not limited to, the methods described below.

In order to make the objectives, technical solutions and advantages of the present disclosure clearer, the present disclosure will be further described in detail below in conjunction with specific examples. It should be understood that the specific examples described here are only used to explain the present disclosure and are not intended to limit the present invention. If no specific technology or conditions are indicated in examples, the technology or conditions described in the literature in the art or the product specification shall be followed. If manufacturers are not indicated for reagents or instruments used, the reagents or instruments are all conventional products that are commercially available. The term “and/or” as used herein includes any and all combinations of one or more related listed items. Examples are provided below to better illustrate the invention, and all temperatures refer to ° C. unless otherwise noted. The names of some compounds in this disclosure are generated by Chemdraw and translated.

### Synthesis of Intermediates

### Preparation of (R,E)-3-(1-methylpyrrolidin-2-yl)acryloyl chloride

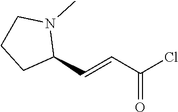

(R,E)-3-(1-methylpyrrolidin-2-yl)acrylic acid (160 mg, 1 mmol) was added to anhydrous dichloromethane (3 ml). Oxalyl chloride (130 mg, 1 mmol) and DMF (1 drop, catalytic amount) were added respectively, and the mixture was stirred at room temperature for 3 hours. The reaction system changed from turbid to clear. The reaction solution was concentrated to give an off-white solid.

### Example 1. (E)-N-(4-((3-chloro-2-fluorophenyl)amino)-5-methoxyquinazolin-6-yl)-4-(dimethylamino)but-2-enamide

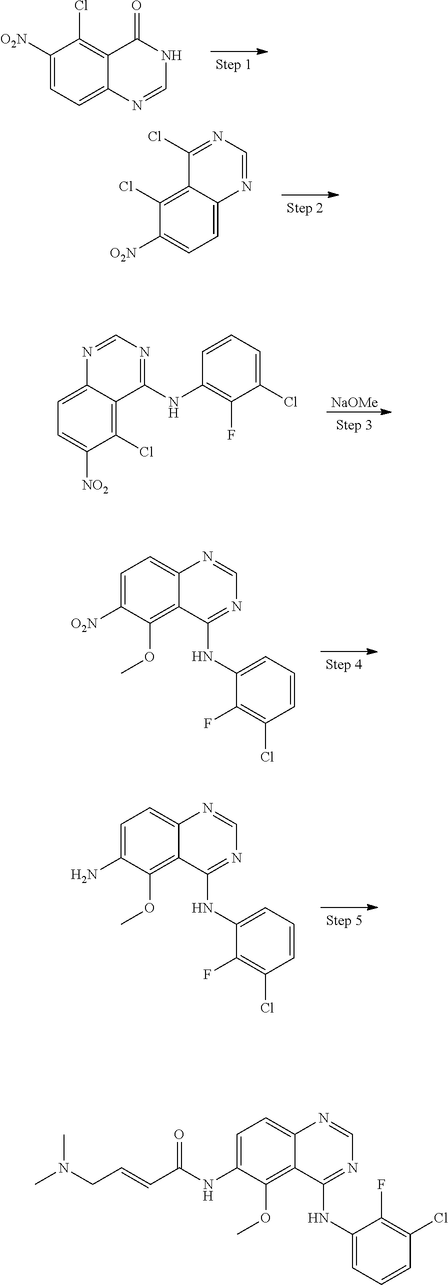

### Step 1): Synthesis of 4,5-dichloro-6-nitroquinazoline

5-chloro-6-nitroquinazolin-4(3H)-one (4.5 g, 20 mmol) was added to dichlorosulfoxide (45 mL), and DMF (2 mL) was added. The mixture was heated to 80° C. and reacted under reflux. After the product was completely dissolved clearly, the reaction solution was reacted for another 2 hours. The reaction solution was concentrated, and toluene was added. The mixture was concentrated again to give 4.9 g of a white solid product;

### Step 2): 5-chloro-N-(3-chloro-2-fluorophenyl)-6-nitroquinazolin-4-amine

4,5-dichloro-6-nitroquinazoline (4.9 g, 20 mmol) was added to anhydrous acetonitrile. 3-chloro-2-fluoroaniline (4.35 g, 30 mmol) and triethylamine (3 g, 30 mmol) were added respectively at 0° C. The mixture was heated to 50° C., and reacted for 5 hours. The reaction solution was cooled, concentrated, and washed with methanol to give 5.3 g of a white solid product, yield 75%; LC-MS:353 [M+H]⁺;

### Step 3): N-(3-chloro-2-fluorophenyl)-5-methoxy-6-nitroquinazolin-4-amine

5-chloro-N-(3-chloro-2-fluorophenyl)-6-nitroquinazoline-4-amine (3.5 g, 10 mmol) was added to a mixed solution of DMF (15 mL) and sodium methoxide solution (a 30% solution of sodium methoxide in methanol, 15 mL) at 0° C. The mixture was reacted with stirring for 2 h, and quenched with ice. The mixture was filtered and dried to give 4.1 g of a yellow solid product, yield 94%. MS:349[M+H]⁺;

### Step 4): N⁴-(3-chloro-2-fluorophenyl)-5-methoxyquinazoline-4,6-diamine

N-(3-chloro-2-fluorophenyl)-5-methoxy-6-nitroquinazoline-4-amine (1.75 g, 5 mmol) was added into ethanol. Iron powder and ammonium chloride aqueous solution were added. The mixture was heated to 50° C. and reacted for 2 hours. The reaction solution was cooled, filtered, and washed with a large amount of dichloromethane. The filtrate was washed with brine, dried and concentrated to give 1.6 g of a light purple solid product, yield 98%; MS:319[M+H]⁺;

Step 5): N⁴-(3-chloro-2-fluorophenyl)-5-methoxyquinazoline-4,6-diamine (32 mg, 0.1 mmol) was added into NMP (1 mL). A solution of (E)-4-(dimethylamino)but-2-enoyl chloride (24 mg, 0.15 mmol) in dichloromethane (1 mL) was added at 0° C. The mixture was reacted with stirring for half an hour. The reaction solution was quenched by adding water. The pH was adjusted to 9 with sodium bicarbonate. The mixture was then extracted with dichloromethane, washed with saturated brine, dried and concentrated. The obtained oil was purified by column chromatography to give 16 mg of a white solid product; ¹H NMR (400 MHz, DMSO-d₆) δ 10.26 (s, 1H), 10.03 (s, 1H), 8.61-8.48 (m, 2H), 8.36 (d, J=9.0 Hz, 1H), 7.61 (d, J=9.1 Hz, 1H), 7.39-7.25 (m, 2H), 6.82 (dt, J=15.5, 5.9 Hz, 1H), 6.59 (d, J=15.5 Hz, 1H), 3.94 (s, 3H), 3.12-3.05 (m, 2H), 2.20 (s, 6H). MS:430 [M+H]⁺.

### Example 2. (E)-N-(4-((3-chloro-2-fluorophenyl)amino)-5-methoxyquinazolin-6-yl)-4-(cyclopropyl(methyl)amino)but-2-enamide

The compound was synthesized using the same method as in Example 1, except that (E)-4-(cyclopropyl(methyl)amino)but-2-enoyl chloride was used to replace (E)-4-(dimethylamino)but-2-enoyl chloride for reaction in step 5; ¹H NMR (400 MHz, DMSO-d₆) δ 10.26 (s, 1H), 10.02 (s, 1H), 8.61-8.50 (m, 2H), 8.37 (d, J=9.1 Hz, 1H), 7.61 (d, J=9.0 Hz, 1H), 7.40-7.25 (m, 2H), 6.86 (dt, J=15.4, 6.2 Hz, 1H), 6.56 (d, J=15.4 Hz, 1H), 3.94 (s, 3H), 3.37-3.29 (m, 2H), 2.29 (s, 3H), 1.76 (tt, J=6.7, 3.5 Hz, 1H), 0.48-0.44 (m, 2H), 0.39-0.31 (m, 2H). MS:456 [M+H]⁺.

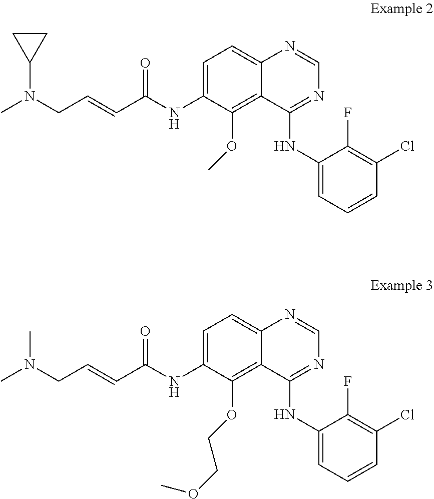 [SMILES: COc1c(NC(=O)/C=C/CN(C)C2CC2)ccc2ncnc(Nc3cccc(Cl)c3F)c12 ; COCCOc1c(NC(=O)/C=C/CN(C)C)ccc2ncnc(Nc3cccc(Cl)c3F)c12]

### Example 3. (E)-N-(4-((3-chloro-2-fluorophenyl)amino)-5-(2-methoxyethoxy)quinazolin-6-yl)-4-(dimethylamino)but-2-enamide

The compound was synthesized using the same method as in Example 1, except that sodium methoxide was replaced with sodium 2-methoxyethanolate for reaction in step 3; ¹H NMR (400 MHz, DMSO-d₆) δ 10.10 (s, 1H), 9.84 (s, 1H), 8.53 (s, 1H), 8.43 (d, J=9.1 Hz, 1H), 8.25-8.16 (m, 1H), 7.63 (d, J=9.0 Hz, 1H), 7.42 (s, 1H), 7.35-7.26 (m, 1H), 6.82 (dt, J=15.4, 5.8 Hz, 1H), 6.45 (d, J=15.5 Hz, 1H), 4.23-4.16 (m, 2H), 3.78-3.71 (m, 2H), 3.20 (s, 3H), 3.12-3.06 (m, 2H), 2.20 (s, 6H). MS:474 [M+H]⁺.

### Example 4. (E)-N-(4-((3-chloro-2-fluorophenyl)amino)-5-(2-fluoroethoxy)quinazolin-6-yl)-4-(dimethylamino)but-2-enamide

The compound was synthesized using the same method as in Example 1, except that sodium methoxide was replaced with sodium 2-fluoroethanolate for reaction in step 3; ¹H NMR (400 MHz, DMSO-d₆) δ 9.97 (d, J=20.0 Hz, 2H), 8.58 (s, 1H), 8.39-8.26 (m, 2H), 7.65 (d, J=9.1 Hz, 1H), 7.39 (ddd, J=8.4, 6.8, 1.7 Hz, 1H), 7.30 (td, J=8.1, 1.4 Hz, 1H), 6.81 (dt, J=15.4, 5.9 Hz, 1H), 6.48 (dt, J=15.4, 1.6 Hz, 1H), 4.91-4.84 (m, 1H), 4.79-4.73 (m, 1H), 4.38 (dd, J=4.7, 2.7 Hz, 1H), 4.34-4.27 (m, 1H), 3.09-3.07 (d, J=5.9 Hz, 2H), 2.19 (s, 6H). MS:462 [M+H]⁺.

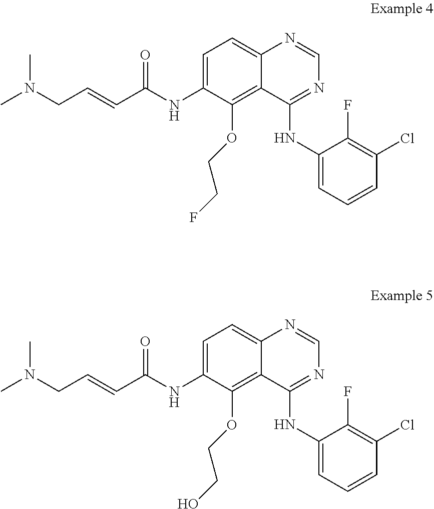 [SMILES: CN(C)C/C=C/C(=O)Nc1ccc2ncnc(Nc3cccc(Cl)c3F)c2c1OCCF ; CN(C)C/C=C/C(=O)Nc1ccc2ncnc(Nc3cccc(Cl)c3F)c2c1OCCO]

### Example 5. (E)-N-(4-((3-chloro-2-fluorophenyl)amino)-5-(2-hydroxyethoxy)quinazolin-6-yl)-4-(dimethylamino)but-2-enamide

The compound was synthesized using the same method as in Example 1, except that sodium methoxide was replaced with sodium 2-hydroxyethanolate for reaction in step 3; ¹H NMR (400 MHz, DMSO-d₆) δ 10.22 (s, 1H), 10.05 (s, 1H), 8.54-8.51 (m, 2H), 8.17 (dd, J=8.4, 7.1 Hz, 1H), 7.62 (d, J=9.1 Hz, 1H), 7.41 (dd, J=8.3, 6.8 Hz, 1H), 7.29 (d, J=8.2 Hz, 1H), 6.82 (dt, J=15.4, 6.0 Hz, 1H), 6.43 (d, J=15.4 Hz, 1H), 5.75 (s, 1H), 4.15-4.13 (m, 2H), 3.85 (s, 2H), 3.08 (d, J=6.0 Hz, 2H), 2.19 (s, 6H). MS:460 [M+H]⁺.

### Example 6. (E)-N-(4-((3-chloro-2-fluorophenyl)amino)-5-(3-methoxypropoxy)quinazolin-6-yl)-4-(dimethylamino)but-2-enamide

The compound was synthesized using the same method as in Example 1, except that sodium methoxide was replaced with sodium 3-methoxypropoxide for reaction in step 3; ¹H NMR (400 MHz, DMSO-d₆) δ 10.12 (s, 1H), 9.90 (s, 1H), 8.59 (s, 1H), 8.43 (dd, J=8.7, 7.3 Hz, 1H), 8.24 (d, J=9.0 Hz, 1H), 7.63 (d, J=9.0 Hz, 1H), 7.42-7.29 (m, 2H), 6.82 (dt, J=15.4, 5.9 Hz, 1H), 6.48 (d, J=15.5 Hz, 1H), 4.11 (t, J=6.4 Hz, 2H), 3.50 (t, J=6.1 Hz, 2H), 3.14 (s, 3H), 3.09 (d, J=6.0 Hz, 2H), 2.20 (s, 6H), 2.09 (q, J=6.3 Hz, 2H). MS:488 [M+H]⁺.

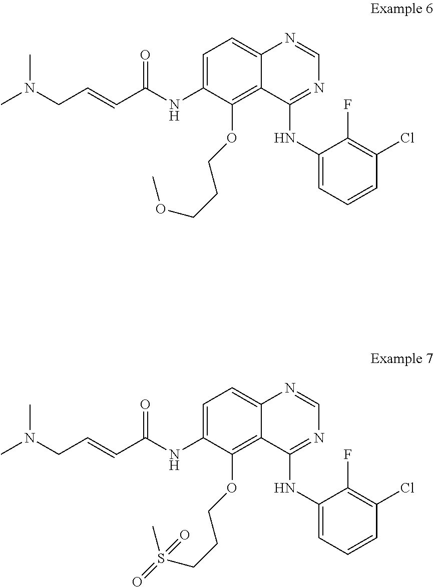 [SMILES: COCCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCCS(C)(=O)=O]

### Example 7. (E)-N-(4-((3-chloro-2-fluorophenyl)amino)-5-(3-(methylsulfonyl)propoxy)quinazolin-6-yl)-4-(dimethylamino)but-2-enamide

The compound was synthesized using the same method as in Example 1, except that sodium methoxide was replaced with sodium 3-methylsulfonylpropoxide for reaction in step 3; ¹H NMR (400 MHz, DMSO-d₆) δ 10.00 (s, 1H), 9.95 (s, 1H), 8.60 (s, 1H), 8.45 (dd, J=8.6, 7.3 Hz, 1H), 8.24 (d, J=9.0 Hz, 1H), 7.65 (d, J=9.0 Hz, 1H), 7.42-7.29 (m, 2H), 6.82 (dt, J=15.4, 5.9 Hz, 1H), 6.51 (d, J=15.7 Hz, 1H), 4.13 (t, J=6.7 Hz, 2H), 3.34-3.26 (m, 2H), 3.09 (d, J=5.9 Hz, 2H), 2.96 (s, 3H), 2.34-2.26 (m, 2H), 2.20 (s, 6H). MS:536 [M+H]⁺.

### Example 8. (E)-N-(4-((3-chloro-2-fluorophenyl)amino)-5-methoxyquinazolin-6-yl)-4-(isopropyl(methyl)amino)but-2-enamide

The compound was synthesized using the same method as in Example 1, except that (E)-4-(dimethylamino)but-2-enoyl chloride was replaced with (E)-4-(isopropyl(methyl)amino)but-2-enoyl chloride for reaction in step 5; ¹H NMR (400 MHz, DMSO-d₆) δ 10.27 (s, 1H), 10.04 (s, 1H), 8.62-8.49 (m, 2H), 8.36 (d, J=9.1 Hz, 1H), 7.62 (d, J=9.1 Hz, 1H), 7.39-7.29 (m, 2H), 6.82 (dt, J=15.4, 5.7 Hz, 1H), 6.60 (d, J=15.4 Hz, 1H), 3.94 (s, 3H), 3.20 (d, J=5.7 Hz, 2H), 2.86-2.80 (m, 1H), 2.15 (s, 3H), 0.99 (d, J=6.5 Hz, 6H). MS:458 [M+H]⁺.

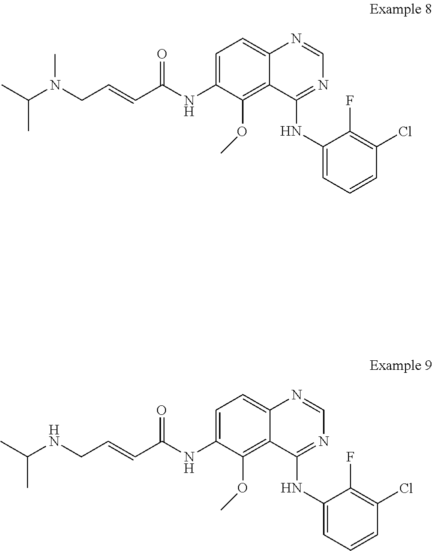 [SMILES: COc1c(NC(=O)/C=C/CN(C)C(C)C)ccc2ncnc(Nc3cccc(Cl)c3F)c12 ; COc1c(NC(=O)/C=C/CNC(C)C)ccc2ncnc(Nc3cccc(Cl)c3F)c12]

### Example 9. (E)-N-(4-((3-chloro-2-fluorophenyl)amino)-5-methoxyquinazolin-6-yl)-4-(isopropylamino)but-2-enamide

The compound was synthesized using the same method as in Example 1, except that (E)-4-(dimethylamino)but-2-enoyl chloride was replaced with (E)-4-(isopropylamino)but-2-enoyl chloride in step 5; ¹H NMR (400 MHz, DMSO-d₆) δ 10.28 (s, 1H), 10.04 (s, 1H), 8.60 (s, 1H), 8.57-8.51 (m, 1H), 8.35 (d, J=9.1 Hz, 1H), 7.63 (d, J=9.1 Hz, 1H), 7.42-7.26 (m, 2H), 6.91 (dt, J=15.4, 5.4 Hz, 1H), 6.60 (dt, J=15.3, 1.8 Hz, 1H), 3.94 (s, 3H), 3.41 (d, J=5.5 Hz, 2H), 2.84-2.78 (m, 1H), 1.04 (d, J=6.2 Hz, 6H). MS:444 [M+H]⁺.

### Example 10. (E)-N-(4-((3-chloro-2-fluorophenyl)amino)-5-isopropoxyquinazolin-6-yl)-4-(dimethylamino)but-2-enamide

The compound was synthesized using the same method as in Example 1, except that sodium isopropoxide was used to replace sodium methoxide for reaction in step 3; ¹H NMR (400 MHz, DMSO-d₆) δ 10.36 (s, 1H), 10.04 (s, 1H), 8.69 (t, J=7.6 Hz, 1H), 8.63 (s, 1H), 8.14 (d, J=9.0 Hz, 1H), 7.62 (d, J=9.0 Hz, 1H), 7.39-7.25 (m, 2H), 6.82 (dt, J=15.5, 5.9 Hz, 1H), 6.49 (d, J=15.5 Hz, 1H), 4.45 (p, J=6.3 Hz, 1H), 3.08 (d, J=6.0 Hz, 2H), 2.19 (s, 6H), 1.32 (d, J=6.2 Hz, 6H). MS:458 [M+H]⁺.

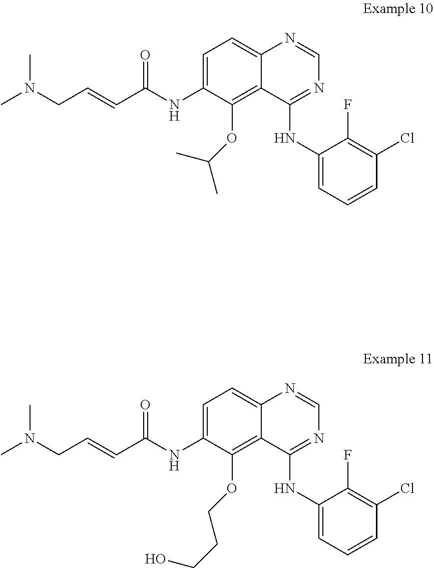 [SMILES: CC(C)Oc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCCO]

### Example 11. (E)-N-(4-((3-chloro-2-fluorophenyl)amino)-5-(3-hydroxypropoxy)quinazolin-6-yl)-4-(dimethylamino)but-2-enamide

The compound was synthesized using the same method as in Example 1, except that sodium methoxide was replaced with sodium 3-hydroxypropoxide for reaction in step 3; ¹H NMR (400 MHz, DMSO-d₆) δ 10.19 (s, 1H), 9.98 (s, 1H), 8.58 (s, 1H), 8.54-8.39 (m, 2H), 7.63 (d, J=9.1 Hz, 1H), 7.42-7.25 (m, 2H), 6.82 (dt, J=15.4, 6.0 Hz, 1H), 6.55-6.46 (m, 1H), 4.98 (s, 1H), 4.15 (t, J=6.4 Hz, 2H), 3.65 (t, J=6.0 Hz, 2H), 3.10 (d, J=6.1 Hz, 2H), 2.20 (s, 6H), 2.01 (q, J=6.1 Hz, 2H). MS:474 [M+H]⁺.

### Example 12. (E)-N-(4-((3-chloro-2-fluorophenyl)amino)-5-(2-(methylthio)ethoxy)quinazolin-6-yl)-4-(dimethylamino)but-2-enamide

The compound was synthesized using the same method as in Example 1, except that sodium methoxide was replaced with sodium 2-methylthioethanolate for reaction in step 3; ¹H NMR (400 MHz, DMSO-d₆) δ 10.05 (s, 1H), 9.94 (s, 1H), 8.55 (s, 1H), 8.30 (d, J=9.1 Hz, 1H), 8.22 (t, J=7.7 Hz, 1H), 7.63 (d, J=9.0 Hz, 1H), 7.42 (t, J=7.5 Hz, 1H), 7.30 (t, J=8.2 Hz, 1H), 6.83 (dt, J=15.5, 5.9 Hz, 1H), 6.50 (d, J=15.4 Hz, 1H), 4.19 (t, J=6.4 Hz, 2H), 3.09 (d, J=5.8 Hz, 2H), 2.98 (t, J=6.3 Hz, 2H), 2.19 (s, 6H), 1.97 (s, 3H). MS:490 [M+H]⁺.

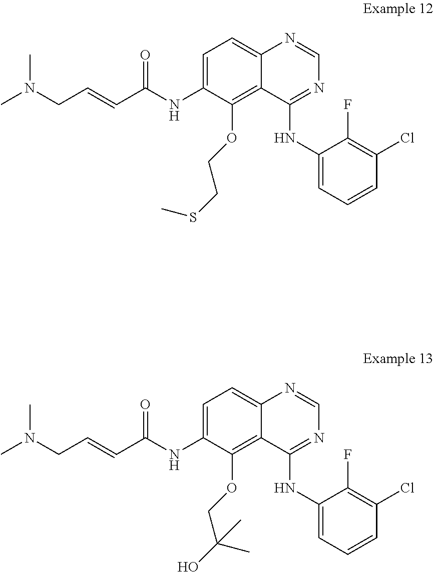 [SMILES: CSCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCC(C)(C)O]

### Example 13. (E)-N-(4-((3-chloro-2-fluorophenyl)amino)-5-(2-hydroxy-2-methylpropoxy)quinazolin-6-yl)-4-(dimethylamino)but-2-enamide

The compound was synthesized using the same method as in Example 1, except that sodium methoxide was replaced with sodium 2-hydroxy-2-methylpropoxide for reaction in step 3; ¹H NMR (400 MHz, DMSO-d₆) δ 10.12 (s, 2H), 8.50 (s, 1H), 8.35 (d, J=9.0 Hz, 1H), 8.01 (t, J=7.5 Hz, 1H), 7.62 (d, J=9.0 Hz, 1H), 7.44 (dd, J=8.3, 6.8 Hz, 1H), 7.29 (t, J=8.1 Hz, 1H), 6.81 (dt, J=15.4, 6.1 Hz, 1H), 6.33 (d, J=15.4 Hz, 1H), 5.66 (s, 1H), 3.87 (s, 2H), 3.08 (dd, J=6.1 Hz, 2H), 2.18 (s, 6H), 1.26 (s, 6H). MS:488 [M+H]⁺.

### Example 14. (E)-N-(4-((3-chloro-2-fluorophenyl)amino)-5-(3-hydroxy-3-methylbutoxy)quinazolin-6-yl)-4-(dimethylamino)but-2-enamide

The compound was synthesized using the same method as in Example 1, except that sodium methoxide was replaced with sodium 3-hydroxy-3-methylbutoxide for reaction in step 3; ¹H NMR (400 MHz, DMSO-d₆) δ 10.25 (s, 2H), 8.69-8.14 (m, 3H), 7.58 (s, 1H), 7.35-7.25 (m, 2H), 6.81 (dt, J=15.4, 6.0 Hz, 1H), 6.54 (d, J=15.4 Hz, 1H), 4.18 (t, J=6.7 Hz, 2H), 3.08 (d, J=6.1 Hz, 2H), 2.19 (s, 6H), 2.00 (t, J=6.8 Hz, 2H), 1.13 (s, 6H). MS:502 [M+H]⁺.

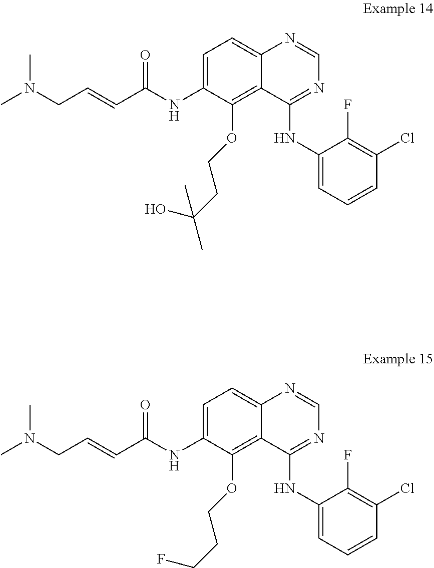 [SMILES: CN(C)C/C=C/C(=O)Nc1ccc2ncnc(Nc3cccc(Cl)c3F)c2c1OCCC(C)(C)O ; CN(C)C/C=C/C(=O)Nc1ccc2ncnc(Nc3cccc(Cl)c3F)c2c1OCCCF]

### Example 15. (E)-N-(4-((3-chloro-2-fluorophenyl)amino)-5-(3-fluoropropoxy)quinazolin-6-yl)-4-(dimethylamino)but-2-enamide

The compound was synthesized using the same method as in Example 1, except that sodium methoxide was replaced with sodium 3-fluoropropoxide for reaction in step 3; ¹H NMR (400 MHz, DMSO-d₆) δ 10.07 (s, 1H), 9.95 (s, 1H), 8.60 (s, 1H), 8.54-8.37 (m, 1H), 8.19 (d, J=9.0 Hz, 1H), 7.64 (d, J=9.0 Hz, 1H), 7.39 (dd, J=8.4, 6.8 Hz, 1H), 7.31 (t, J=8.2 Hz, 1H), 6.81 (dt, J=15.5, 5.9 Hz, 1H), 6.47 (d, J=15.5 Hz, 1H), 4.72 (t, J=5.8 Hz, 1H), 4.60 (t, J=5.8 Hz, 1H), 4.14 (t, J=6.5 Hz, 2H), 3.08 (dd, J=5.9 Hz, 2H), 2.31-2.15 (m, 2H), 2.19 (s, 6H). MS:476 [M+H]⁺.

### Example 16. (E)-N-(4-((3-chloro-2-fluorophenyl)amino)-5-(2,2,2-trifluoroethoxy)quinazolin-6-yl)-4-(dimethylamino)but-2-enamide

The compound was synthesized using the same method as in Example 1, except that sodium methoxide was replaced with sodium 2,2,2-trifluoroethanolate for reaction in step 3; ¹H NMR (400 MHz, DMSO-d₆) δ 10.04 (s, 1H), 9.59 (s, 1H), 8.61 (d, J=8.0 Hz, 1H), 8.39 (s, 1H), 8.14 (d, J=9.0 Hz, 1H), 7.69 (d, J=9.0 Hz, 1H), 7.43-7.35 (m, 1H), 7.31 (t, J=8.2 Hz, 1H), 6.81 (dt, J=15.5, 5.9 Hz, 1H), 6.43 (d, J=15.5 Hz, 1H), 4.77 (q, J=9.0 Hz, 2H), 3.09 (d, J=6.0 Hz, 2H), 2.20 (s, 6H). MS:498 [M+H]⁺.

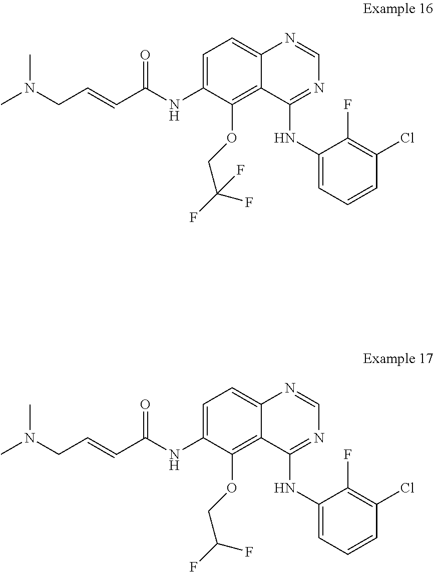 [SMILES: CN(C)C/C=C/C(=O)Nc1ccc2ncnc(Nc3cccc(Cl)c3F)c2c1OCC(F)(F)F ; CN(C)C/C=C/C(=O)Nc1ccc2ncnc(Nc3cccc(Cl)c3F)c2c1OCC(F)F]

### Example 17. (E)-N-(4-((3-chloro-2-fluorophenyl)amino)-5-(2,2-difluoroethoxy)quinazolin-6-yl)-4-(dimethylamino)but-2-enamide

The compound was synthesized using the same method as in Example 1, except that sodium methoxide was replaced with sodium 2,2-difluoroethanolate for reaction in step 3; ¹H NMR (400 MHz, DMSO-d₆) δ 10.01 (s, 1H), 9.74 (s, 1H), 8.58 (s, 1H), 8.33-8.24 (m, 1H), 8.19 (d, J=9.0 Hz, 1H), 7.66 (d, J=9.0 Hz, 1H), 7.42 (dd, J=8.4, 6.7 Hz, 1H), 7.31 (t, J=8.2 Hz, 1H), 6.82 (dt, J=15.4, 5.9 Hz, 1H), 6.63-6.30 (m, 2H), 4.38 (t, J=15.4 Hz, 2H), 3.12-3.06 (m, 2H), 2.20 (s, 6H). MS:480 [M+H]⁺.

### Example 18. (E)-N-(4-((3-chloro-2-fluorophenyl)amino)-5-(3,3,3-trifluoropropoxy)quinazolin-6-yl)-4-(dimethylamino)but-2-enamide

The compound was synthesized using the same method as in Example 1, except that sodium methoxide was replaced with sodium 3,3,3-trifluoropropoxide for reaction in step 3; ¹H NMR (400 MHz, DMSO-d₆) δ 9.93 (s, 1H), 9.73 (s, 1H), 8.55 (s, 1H), 8.17 (s, 2H), 7.64 (d, J=9.2 Hz, 1H), 7.43 (t, J=7.5 Hz, 1H), 7.30 (t, J=8.4 Hz, 1H), 6.81 (d, J=15.3 Hz, 1H), 6.48 (d, J=15.4 Hz, 1H), 4.24 (t, J=6.4 Hz, 2H), 3.11-3.05 (m, 2H), 3.04-2.96 (m, 2H), 2.19 (s, 6H). MS:512 [M+H]⁺.

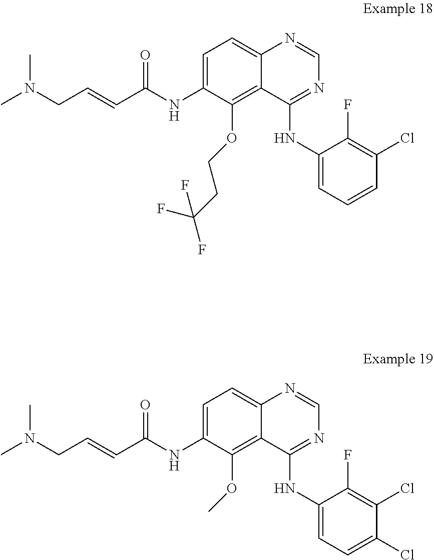 [SMILES: CN(C)C/C=C/C(=O)Nc1ccc2ncnc(Nc3cccc(Cl)c3F)c2c1OCCC(F)(F)F ; COc1c(NC(=O)/C=C/CN(C)C)ccc2ncnc(Nc3ccc(Cl)c(Cl)c3F)c12]

### Example 19. (E)-N-(4-((3,4-dichloro-2-fluorophenyl)amino)-5-methoxyquinazolin-6-yl)-4-(dimethylamino)but-2-enamide

The compound was synthesized using the same method as in Example 1, except that 3-chloro-2-fluoroaniline was replaced with 3,4-dichloro-2-fluoroaniline for reaction in step 2; ¹H NMR (400 MHz, DMSO-d₆) δ 10.27 (s, 1H), 10.06 (s, 1H), 8.63-8.53 (m, 2H), 8.38 (d, J=9.1 Hz, 1H), 7.62 (t, J=8.7 Hz, 2H), 6.82 (dt, J=15.5, 5.8 Hz, 1H), 6.59 (d, J=15.4 Hz, 1H), 3.93 (s, 3H), 3.10 (d, J=5.7 Hz, 2H), 2.21 (s, 6H). MS:464 [M+H]⁺.

### Example 20. (R,E)-N-(4-((3,4-dichloro-2-fluorophenyl)amino)-5-methoxyquinazolin-6-yl)-3-(1-methylpyrrolidin-2-yl)acrylamide

The compound was synthesized using the same method as in Example 1, except that 3-chloro-2-fluoroaniline was replaced with 3,4-dichloro-2-fluoroaniline in step 2, and (E)-4-(dimethylamino)but-2-enoyl chloride was replaced with (R,E)-3-(1-methylpyrrolidin-2-yl)acryloyl chloride in step 5 for reaction; ¹H NMR (400 MHz, DMSO-d₆) δ 10.27 (s, 1H), 10.04 (s, 1H), 8.63-8.53 (m, 2H), 8.39 (d, J=9.1 Hz, 1H), 7.67-7.58 (m, 2H), 6.71 (dd, J=15.3, 7.5 Hz, 1H), 6.57 (d, J=15.3 Hz, 1H), 3.94 (s, 3H), 3.04 (dd, J=9.8, 7.1 Hz, 1H), 2.76 (q, J=7.9 Hz, 1H), 2.22 (s, 3H), 2.23-2.13 (m, 1H), 2.02 (dtd, J=12.3, 8.4, 5.9 Hz, 1H), 1.82-1.68 (m, 2H), 1.66-1.52 (m, 1H). MS:490 [M+H]⁺.

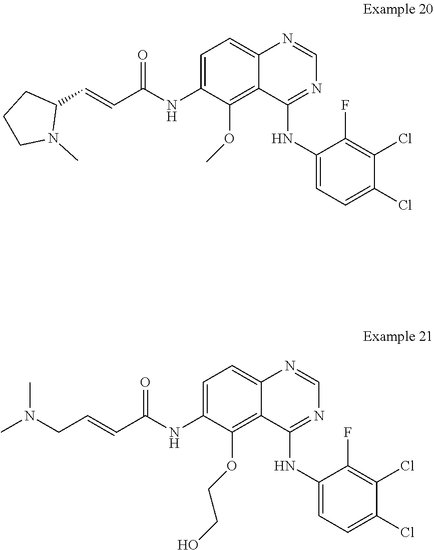

### Example 21. (E)-N-(4-((3,4-dichloro-2-fluorophenyl)amino)-5-(2-hydroxyethoxy)quinazolin-6-yl)-4-(dimethylamino)but-2-enamide

The compound was synthesized using the same method as in Example 1, except that 3,4-dichloro-2-fluoroaniline was used to replace 3-chloro-2-fluoroaniline in step 2, and sodium 2-hydroxyethanolate was used to replace sodium methoxide in step 3 for reaction; ¹H NMR (400 MHz, DMSO-d₆) δ 10.24 (s, 1H), 10.05 (s, 1H), 8.58-8.49 (m, 2H), 8.23 (t, J=8.5 Hz, 1H), 7.67-7.56 (m, 2H), 6.82 (dt, J=15.4, 6.0 Hz, 1H), 6.43 (dt, J=15.4 Hz, 1H), 5.75-5.68 (m, 1H), 4.15-4.13 (m, 2H), 3.84 (q, J=4.3 Hz, 2H), 3.10 (dd, J=6.0 Hz, 2H), 2.20 (s, 6H). MS:494 [M+H]⁺.

### Example 22. (R,E)-N-(4-((3,4-dichloro-2-fluorophenyl)amino)-5-(2-hydroxyethoxy)quinazolin-6-yl)-3-(1-methylpyrrolidin-2-yl)acrylamide

The compound was synthesized using the same method as in Example 1, except that 3,4-dichloro-2-fluoroaniline was used to replace 3-chloro-2-fluoroaniline in step 2, sodium methoxide was replaced with sodium 2-hydroxyethanolate in step 3, and (R,E)-3-(1-methylpyrrolidin-2-yl)acryloyl chloride was used to replace (E)-4-(dimethylamino)but-2-enoyl chloride in step 5 for reaction; ¹H NMR (400 MHz, DMSO-d₆) δ 10.24 (s, 1H), 10.04 (s, 1H), 8.54 (d, J=9.6 Hz, 2H), 8.24 (t, J=8.5 Hz, 1H), 7.67-7.56 (m, 2H), 6.71 (dd, J=15.3, 7.8 Hz, 1H), 6.41 (d, J=15.3 Hz, 1H), 5.73 (t, J=4.3 Hz, 1H), 4.14 (dd, J=5.2, 3.3 Hz, 2H), 3.85 (q, J=4.3 Hz, 2H), 3.04 (dd, J=9.8, 7.3 Hz, 1H), 2.76 (q, J=8.0 Hz, 1H), 2.21 (s, 3H), 2.18 (t, J=8.8 Hz, 1H), 2.08-1.94 (m, 1H), 1.82-1.68 (m, 2H), 1.66-1.52 (m, 1H). MS:520 [M+H]⁺.

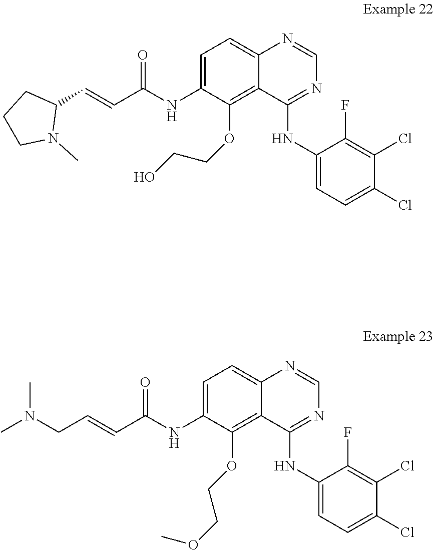

### Example 23. (E)-N-(4-((3,4-dichloro-2-fluorophenyl)amino)-5-(2-methoxyethoxy)quinazolin-6-yl)-4-(dimethylamino)but-2-enamide

The compound was synthesized using the same method as in Example 1, except that 3,4-dichloro-2-fluoroaniline was used to replace 3-chloro-2-fluoroaniline in step 2, and sodium 2-methoxyethanolate was used to replace sodium methoxide in step 3 for reaction; ¹H NMR (400 MHz, DMSO-d₆) δ 9.85 (br, 2H), 8.43 (d, J=9.1 Hz, 1H), 8.37 (s, 1H), 8.08 (s, 1H), 7.51 (d, J=9.0 Hz, 2H), 6.80 (dt, J=15.4, 5.8 Hz, 1H), 6.39 (d, J=15.4 Hz, 1H), 4.26-4.19 (m, 2H), 3.75-3.68 (m, 2H), 3.25 (s, 3H), 3.08 (dd, J=5.8, 1.7 Hz, 2H), 2.19 (s, 6H). MS:508 [M+H]⁺.

### Example 24. (R,E)-N-(4-((3,4-dichloro-2-fluorophenyl)amino)-5-(2-methoxyethoxy)quinazolin-6-yl)-3-(1-methylpyrrolidin-2-yl)acrylamide

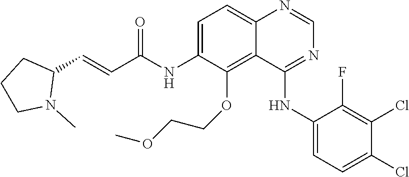

The compound was synthesized using the same method as in Example 1, except that 3,4-dichloro-2-fluoroaniline was used to replace 3-chloro-2-fluoroaniline in step 2, sodium methoxide was replaced with sodium 2-methoxyethanolate in step 3, and (R,E)-3-(1-methylpyrrolidin-2-yl)acryloyl chloride was used to replace (E)-4-(dimethylamino)but-2-enoyl chloride in step 5 for reaction; ¹H NMR (400 MHz, DMSO-d₆) δ 10.11 (s, 1H), 9.84 (s, 1H), 8.54 (s, 1H), 8.45 (d, J=9.1 Hz, 1H), 8.29 (t, J=8.5 Hz, 1H), 7.67-7.56 (m, 2H), 6.71 (dd, J=15.3, 7.6 Hz, 1H), 6.43 (d, J=15.3 Hz, 1H), 4.23-4.16 (m, 2H), 3.74 (d, J=6.0 Hz, 2H), 3.20 (s, 3H), 3.04 (dd, J=9.7, 7.0 Hz, 1H), 2.78 (q, J=7.9 Hz, 1H), 2.22 (s, 3H), 2.19 (q, J=8.8 Hz, 1H), 2.08-1.95 (m, 1H), 1.81-1.68 (m, 2H), 1.65-1.51 (m, 1H). MS:534 [M+H]⁺.

### Assay Example 1. Assay of Inhibition of EGFR^{WT} and HER2 Kinase Activity by Small Molecule Compounds

Reagents and consumables: ULight™-labeled Ploy GT Peptide (Perkin Elmer, Cat. No. TRF-0100-M); ULight™-labeled JAK-1 (Try1023) Peptide (Perkin Elmer, Cat. No. TRF-0121-M); Eu-W1024-labeled Anti-Phosphotyrosine Antibody (PT66) (Perkin Elmer, Cat. No. AD0068); 10×Detection Buffer (Perkin Elmer, Cat. No. CR97-100); HER2 Kinase (Carna Biosciences, Cat. No. 08-016); EGFR kinase (Carna Biosciences, Cat. No. 08-115); HEPES (GIBCO, Cat. No. 15630-080); EGTA (Sigma, Cat. No. 03777-10G); EDTA (Sigma, Cat. No. EDS-100G); MgCl₂ (Sigma, Cat. No. 63069-100ML); DTT (Sigma, Cat. No. 43816-10ML); Tween-20 (Sigma, Cat. No. P7949-100ML); DMSO (Life Science, Cat. No. 0231-500ML); 384-well plates (Perkin Elmer, Cat. No. 607290); multifunctional plate reader (Perkin Elmer, Cat. No. Envision)

Preparation of compound solution: The assay compound was dissolved in DMSO to form a 10 mM stock solution. Prior to use, the compound was diluted to 0.25 mM in DMSO (100 times the final concentration of the diluent) and then subjected to a 3-fold concentration gradient dilution to obtain a total of 11 concentrations. The solution was diluted with buffer to a diluent with 4× the final concentration when the drug was added.

HER2 kinase assay: buffer was prepared, and 40 nM 4× HER2 kinase solution, 40 μM 4× ATP solution, and 400 nM 4×ULight™-labeled Ploy GT Peptide substrate solution were prepared using the buffer. After the preparation was completed, the enzyme was mixed with different concentrations of compounds pre-prepared by dilution, and the mixture was left to stand at room temperature for 5 min. Each concentration was performed in duplicate. The corresponding substrate and ATP were added and the mixture was reacted at room temperature for 120 minutes (with negative and positive controls). After completion of the reaction, PT66 detection antibody was added. The mixture was incubated at room temperature for 60 minutes, and then detected by Envision.

EGFR^{WT} kinase assay: buffer was prepared, and 3.48 nM 4× EGFR kinase solution, 600 μM 4× ATP solution, and 400 nM 4×ULight™-labeled JAK-1 (Try1023) Peptide substrate solution was prepared using the buffer. After the preparation was completed, the enzyme was mixed with different concentrations of compounds pre-prepared by dilution, and the mixture was left to stand at room temperature for 5 min. Each concentration was performed in duplicate. The corresponding substrate and ATP were added and the mixture was reacted at room temperature for 120 minutes (with negative and positive controls). After completion of the reaction, PT66 detection antibody was added. The mixture was incubated at room temperature for 60 minutes, and then detected by Envision.

Data Calculation: the reading of well and inhibition rate were calculated using Excel, wherein reading of well=10000*(well EU665 value)/(well EU615 value), and inhibition rate=[(reading of positive control well−reading of assay well)/(reading of positive control well−reading of negative control well)]*100%. Compound concentrations and corresponding inhibition rates were input into GraphPad Prism for processing to calculate IC₅₀ values.

The data in Table 1 show that the compounds of the present application can inhibit the activity of EGFR^{WT} and HER2 tyrosine kinases, especially some of the compounds show strong inhibitory effects. The assay results are summarized in Table 1 below.

The results of the assay of the inhibitory activity of some of the compounds of the present application against EGFR^{WT} and HER2 tyrosine kinases are listed in Table 1, wherein A indicates that the IC₅₀ is less than or equal to 1 nM, B indicates that the IC₅₀ is greater than 1 nM and less than or equal to 10 nM, C indicates that the IC₅₀ is greater than 10 nM and less than or equal to 100 nM, D indicates that the IC₅₀ is greater than 100 nM and less than or equal to 1000 nM, and NT indicates that there is no relevant result.

TABLE 1
Results of the assay of the inhibitory activity
of the compounds of the present disclosure
against EGFR and HER2 kinases

| Example | EGFR^{WT} | HER2 |
|---|---|---|
| No. | IC₅₀ nM | IC₅₀ nM |
| 1 | A | D |
| 2 | B | D |
| 3 | A | C |
| 4 | A | C |
| 5 | A | C |
| 6 | A | D |
| 7 | A | D |
| 8 | A | C |
| 9 | A | C |
| 10 | B | D |
| 11 | A | D |
| 12 | A | C |
| 13 | A | C |
| 14 | B | C |
| 15 | A | C |
| 16 | A | C |
| 17 | A | D |
| 18 | A | D |
| 19 | A | C |
| 20 | A | B |
| 21 | A | C |
| 22 | A | B |
| 23 | A | C |
| 24 | A | C |

From the results in Table 1 above, it can be seen that the compounds of the present application exhibit good to excellent inhibitory activity against HER2 and EGFR kinases, wherein the compounds of the present application exhibit very strong inhibitory activity against EGFR, thus showing a certain selectivity relative to HER2 kinase.

### Assay Example 2. Assay of Inhibition of Cell Proliferation by Small Molecule Compounds

The present application examined the in vitro anti-proliferative activity of the compounds of the present disclosure against in vitro cultured HCC-827, Ba/F3-EGFR-VIII and Ba/F3 EGFR D770_N771insSVD cell lines by CTG method.

Reagents and consumables: RPMI1640 (ThermoFisher, Cat. No. C11875500BT); DMEM (ThermoFisher, C₁₁₉₉₅₅₀₀BT); fetal bovine serum (Hyclone, Cat. No. SV30087.03); 0.25% trypsin-EDTA (ThermoFisher, Cat. No. 25200072); penicillin-streptomycin (Hyclone, Cat. No. SV30010); DMSO (Life Science, Cat. No. 0231-500ML); CTG assay kit (Promega, Cat. No. G9243); 96-well plate (Corning, Cat. No. 3599); multifunctional plate reader (Perkin Elmer, Cat. No. Envision)

Cell lines: HCC-827 (from ATCC), Ba/F3-EGFR-VIII and Ba/F3 EGFR D770_N771insSVD (all from KYinno Biotechnology (Beijing) Co., Ltd.); all of the above cells were cultured with RPMI1640 medium containing 10% fetal bovine serum, 100 U/mL penicillin, and 100 μg/mL streptomycin in the culture process, and HCC-827 was cultured with DMEM medium containing 10% fetal bovine serum, 100 U/mL penicillin, and 100 μg/mL streptomycin.

### Specific Assay Methods:

1. The assay compound was dissolved with DMSO to form a stock solution and a gradient dilution was performed, followed by dilution with the corresponding medium to obtain a solution with 5-fold working concentration.
2. Cells in logarithmic growth phase were adjusted to specific cell densities by dilution with culture medium, and 80 μL of cell suspension was added to a 96-well plate, such that plating densities of cells of HCC-827, Ba/F3-EGFR-VIII and Ba/F3 EGFR D770_N771insSVD were all 3000 cells/well. The cells of Ba/F3-EGFR-VIII and Ba/F3 EGFR D770_N771insSVD were used directly in the next step (treatment with compound), whereas the HCC-827 needed to be cultured overnight in a 37° C., 5% carbon dioxide gas incubator to adhere to the wall, and then treated with compounds.
3. 20 μL of compound solution was added to each well of the 96-well plate that had been inoculated with cells. The highest concentration of the assayed compound was 10 μM, 4-fold serial dilution, a total of 9 concentrations, and duplicate wells. A control group without compound was also set up.
4. The cells were cultured for another 72 hours, and then cell viability was detected by CTG assay kit. Signal values were read with a multifunctional plate reader (Perkin Elmer). GraphPad Prism software was used to create a dose-response curve and IC₅₀ was calculated.

The results of the assay of the anti-proliferative activity of the representative compounds of the present disclosure against cells of HCC-827, Ba/F3-EGFR-VIII and Ba/F3 EGFR D770_N771insSVD are listed in Table 2, wherein A indicates that the IC₅₀ is less than or equal to 5 nM, B indicates that the IC₅₀ is greater than 5 nM and less than or equal to 50 nM, C indicates that the IC₅₀ is greater than 50 nM and less than or equal to 500 nM, D indicates that the IC₅₀ is greater than 500 nM and less than or equal to 5000 nM, and NT indicates that there is no relevant result.

TABLE 2
Results of the assay of the anti-proliferative activity of the
representative compounds of the present disclosure against
the cells of HCC-827, Ba/F3-EGFR-VIII and Ba/F3 EGFR
D770_N771insSVD.

| Example | HCC-827 | Ba/F3 EGFR D770_N771insSVD | Ba/F3 EGFRvIII |
|---|---|---|---|
| No. | IC₅₀ | IC₅₀ | IC₅₀ |
| 1 | A | C | A |
| 2 | A | C | B |
| 3 | A | C | A |
| 4 | A | C | B |
| 5 | A | B | A |
| 6 | A | C | B |
| 7 | A | NT | B |
| 8 | A | C | B |
| 9 | A | B | A |
| 10 | B | C | C |
| 11 | A | C | A |
| 12 | B | NT | B |
| 13 | A | C | A |
| 14 | A | C | B |
| 15 | A | C | B |
| 16 | A | C | B |
| 17 | A | C | B |
| 18 | A | C | B |
| 19 | A | C | B |
| 20 | A | C | NT |
| 21 | A | B | NT |
| 22 | A | B | NT |
| 23 | A | C | NT |
| 24 | A | C | NT |

The results in Table 2 show that the compounds of the present application exhibit a certain anti-tumor proliferation activity against each of the cell lines assayed above. Especially for HCC-827 and Ba/F3-EGFR-VIII cell lines, the compounds of the present application show excellent activity.

### Assay Example 3. Assay of Pharmacokinetics of Small Molecule Compounds

In this assay, the pharmacokinetic characteristics and ability to penetrate the blood-brain barrier of the compounds of the present application were assayed after administration of some of the compounds of the present application to SD rats by single oral administration and intravenous injection.

### (i) Reagents, Instruments and Animals Used

TABLE 3
Assay reagents

| Reagent | Supplier | Cat. No. | Lot No. |
|---|---|---|---|
| DMSO | Innochem | D3851 | INAH03010 |
| Kolliphor ®HS 15 (Solutol) | SIGMA | 42966 | BCBZ6212 |
| Acetonitrile | Fisher | 75-05-8 | 186342 |
| Formic acid | DIKMA | 64-18-6 | 2486917 |
| Ammonium acetate | SIGMA | 431311 | MKCD5084 |
| Ultra-pure water | Manufactured | — | — |
| | in-house | | |

TABLE 4
Assay instruments

| Instrument | Brand | Model |
|---|---|---|
| Liquid chromatography-mass | AB Sciex | 5500 Q-trap |
| spectrometry (LC-MS) | | |
| High speed refrigerated centrifuge | Thermo | ST 40R |
| Balance (centi milli-) | Mettler | XPE205 |
| Single well pipette | Eppendorf | Research plus series |
| Electric 12-hole pipette | Thermo | Novus series |

TABLE 5
Rats used in the assay

| | |
|---|---|
| Animal & strain: | SD Rat (Male) |
| Animal grade: | SPF grade |
| Animal source: | Beijing HFK BioSCIENCE Co., Ltd |
| Weight range, age: | 180 g-350 g on the first day of administration, |
| | 8 weeks old (based on body weight) |

### (ii) Preparation of Sample Formulation

1. Intravenous (IV) injection group: an appropriate amount of the assay compound was weighed, and completely dissolved in an appropriate volume of vehicle (DMSO/Solutol/H₂O=5/10/85 V/V/V with 2 times mole of HCl). The mixture was stirred, vortexed and/or sonicated. Once a solution was obtained, the vehicle was gradually added to a final volume to reach the target concentration. The solution was vortexed, and sonicated to obtain a homogeneous solution. The solution was filtered through a 0.22 μm PVDF membrane.
2. Oral (PO) group: an appropriate amount of the assay compound was weighed, and completely dissolved in an appropriate volume of vehicle (DMSO/Solutol/H₂O=5/10/85 V/V/V with 2 times mole of HCl). The mixture was stirred, vortexed and/or sonicated. Once a solution was obtained, the vehicle was gradually added to a final volume to reach the target concentration. The solution was vortexed, and sonicated to obtain a homogeneous solution.
   (iii) Administration and Sampling of Rats

The animals were randomly divided into groups according to their body weights, and the weights of the animals in each group were comparable after grouping (not exceeding ±20% of the average body weight). Meanwhile, the IV group was not fasted and the PO group was fasted overnight (>12 hrs) and given food 2 hrs after drug administration. All animals had free access to water. The dosing regimen and pharmacokinetic sampling regimen are given in Table 6 and Table 7 below, respectively.

TABLE 6
Dosing regimen

| | | Number | | Dose of | Volume of | Concentration |
|---|---|---|---|---|---|---|
| | Method of | of | | administration | administration | of formulation |
| Group | administration | animals | Sample | (mg/kg) | (mL/kg) | (mg/mL) |
| 1 | IV | 3 | Whole | 3 | 3 | 1 |
| | | | blood | | | |
| 2 | PO | 3 | Whole | 10 | 10 | 1 |
| | | | Blood | | | |
| 3 | PO | 3 | Whole | 10 | 10 | 1 |
| | | | blood/brain | | | |

TABLE 7
Pharmacokinetic sampling regimen

| | Method of | | | Pharmacokinetic time |
|---|---|---|---|---|
| Group | administration | Sampling | Anticoagulant | point |
| 1 | IV | Jugular Vein Cannulation (JVC) | K₂EDTA | Whole blood: pre- |
| | | | | dose, 5, 15, 30 |
| | | | | minutes, 1, 2, 4, 7 and |
| | | | | 24 hours post-dose |
| 2 | PO | Jugular Vein Cannulation (JVC) | K₂EDTA | Whole blood: pre- |
| | | | | dose, 15, 30 minutes, |
| | | | | 1, 2, 4, 7 and 24 hours |
| | | | | post-dose |
| 3 | PO | Blood was collected from the | K₂EDTA | Whole blood: 2 hours |
| | | heart, the animal was | | Brain tissue: 2 hours |
| | | anesthetized, and brain tissue | | |
| | | was collected. | | |

Rats were administered according to the above regimen, and blood and brain tissue samples were collected and processed at predetermined time points (collection and processing were performed according to conventional methods in the art).

### (iv) Analysis of Samples

Brains were weighed and homogenized by adding 4-fold homogenizing solution (acetonitrile/water=1/1). Six times volume of acetonitrile was added respectively to the whole blood samples and brain homogenate. The mixture was vortexed for 1 min and then centrifuged at 4° C., 4500 rpm for 15 min. The supernatant was diluted 2-fold with ultra-pure water, and the samples were analyzed by LC/MS.

### (v) Data Analysis:

Pharmacokinetic parameters were calculated using WinNonlin software. The following pharmacokinetic parameters were calculated if appropriate plasma drug concentration-time data were available: CL (clearance); V_{d} (apparent volume of distribution); T_{1/2} (elimination half-life time); Cₘₐₓ (peak concentration); Tₘₐₓ (time to peak); AUC (area under plasma drug concentration-time curve); MRT (mean retention time); F % (bioavailability).

The assay results are shown in Tables 8-10 below, which respectively provide the rat plasma drug concentration of Example Compound 1 of the present application at each time point, as well as the pharmacokinetic parameter values, and the concentration and concentration ratio of Example Compound 1 of the present application in the brain and blood of rats. It can be seen from the above results that Compound 1 of the present application exhibits excellent ability to penetrate the blood-brain barrier. This also shows that the compounds of the present application not only have excellent EGFR kinase inhibitory activity, and can inhibit cell proliferation at the cellular level, but also have excellent ability to penetrate the blood-brain barrier, and are expected to be used in EGFR kinase-mediated related diseases, especially diseases related to brain metastasis.

TABLE 8
Plasma drug concentration of Example Compound 1 of the
present application in rats

| | Plasma drug concentration of | |
|---|---|---|
| Time point | Example 1 (ng/mL) | |
| (hr) | IV 3 mg/kg | PO 10 mg/kg |
| 0 | 0 | 0 |
| 0.083 | 888 ± 106 | / |
| 0.25 | 494 ± 121 | 83.2 ± 29.9 |
| 0.5 | 365 ± 48 | 227 ± 58 |
| 1 | 175 ± 23 | 281 ± 73 |
| 2 | 78.2 ± 28.7 | 249 ± 74 |
| 4 | 18.8 ± 8.7 | 77.7 ± 7.3 |
| 7 | 7.05 ± 3.77 | 72.6 ± 25.5 |
| 24 | NA | 14.2 ± 19.7 |

TABLE 9
Pharmacokinetic parameters of the compound of Example 1
in the presnt application in rats

| | Example 1 | |
|---|---|---|
| Parameters | IV 3 mg/kg | PO 10 mg/kg |
| Tₘₐₓ (hr) | / | 1.00 ± 0.00 |
| Cₘₐₓ (ng/mL) | / | 281 ± 73 |
| T_{1/2} (hr) | 1.45 ± 0.09 | 3.17 ± 1.22 |
| AUC₀₋ₜ (hr*ng/mL) | 709 ± 96 | 1732 ± 170 |
| AUC_{inf} (hr*ng/mL) | 724 ± 105 | 1776 ± 178 |
| Cl_obs (mL/min/kg) | 70.0 ± 9.5 | / |
| V_{d} (L/kg) | 8.76 ± 0.67 | / |
| F % | / | 73.6 ± 7.4 |

TABLE 10
Concentration and concentration ratio of Example Compound
1 of the present application in brain and whole blood (PO
10 mg/kg, sampling time, 2 hours after administration)

| | Plasma drug | Brain concentration | Brain/ |
|---|---|---|---|
| Example | concentration (ng/mL) | (ng/g) | blood ratio |
| 1 | 480 | 1468 | 3.06 |

The above-mentioned embodiments are alternative embodiments of the present disclosure. It should be pointed out that for those skilled in the art, without departing from the principles of the present disclosure, several improvements and modifications can also be made to the embodiments of the present disclosure, and these improvements and modifications should also be regarded as within the protection scope of the present disclosure.

1. A compound represented by formula (I), a stereoisomer thereof, and a pharmaceutically acceptable salt thereof, 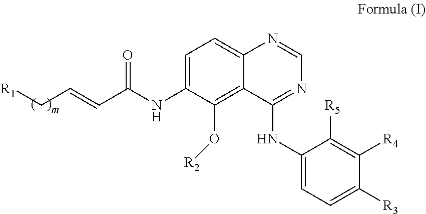
in formula (I), m is 0, 1 or 2;
R₁ is hydrogen, 4- to 7-membered heteroalicyclyl or —NR^{a}R^{b},
R^{a} and R^{b} are each independently hydrogen, C₁-C₆ alkyl, C₃-C₆ cycloalkyl, hydroxy-substituted C₁-C₆ alkyl, C₁-C₃ alkoxy-substituted C₁-C₆ alkyl, or C₃-C₆ cycloalkyl-substituted C₁-C₆ alkyl, the 4- to 7-membered heteroalicyclyl group is a heteroalicyclyl group containing 1-2 heteroatoms selected from N, O and S, wherein the heteroalicyclyl group is unsubstituted or substituted with one or two of C₁-C₃ alkyl, C₁-C₄ acyl, hydroxyl, cyano, aminoacyl, mono- or di-C₁-C₃ alkyl-substituted aminoacyl, C₁-C₃ alkylsulfonyl, C₁-C₃ alkyl sulfoxide group, and oxo (═O);
R₂ is C₁-C₆ alkyl, which is unsubstituted or substituted with 1 to 3 substituents selected from halogen, cyano, C₁-C₃ alkyl, C₁-C₃ alkoxy, C₁-C₃ alkylthio, hydroxyl, C₃-C₄ cycloalkyl and C₁-C₃ alkylsulfonyl;
R₃, R₄ and R₅ are each independently hydrogen, halogen, C₁-C₃ alkyl, C₁-C₃ alkoxy, or C₃-C₄ cycloalkyl, and at least one of R₃, R₄ and R₅ is halogen.

2. The compound according to claim 1, a stereoisomer thereof, and a pharmaceutically acceptable salt thereof, wherein
m is 0 or 1,
R₁ is 4- to 7-membered heteroalicyclyl or —NR^{a}R^{b},
R^{a} and R^{b} are each independently hydrogen, C₁-C₃ alkyl, C₃-C₆ cycloalkyl, hydroxy-substituted C₁-C₃ alkyl, or C₁-C₃ alkoxy-substituted C₁-C₃ alkyl;
the 4- to 7-membered heteroalicyclyl group is pyrrolidinyl, piperidinyl, piperazinyl, morpholinyl, tetrahydrofuranyl, tetrahydropyranyl, or thiomorpholinyl, and the above groups are unsubstituted or substituted with one or two of methyl, ethyl, propyl, isopropyl, aldehyde group, acetyl, propionyl, hydroxy, cyano, aminoacyl, methyl sulfonyl, ethyl sulfonyl, propyl sulfonyl, isopropyl sulfonyl, methyl sulfoxide group, ethyl sulfoxide group, propyl sulfoxide group, isopropyl sulfoxide group, and oxo (═O).

3. The compound according to claim 2, a stereoisomer thereof, and a pharmaceutically acceptable salt thereof, wherein R₁ is 1-methylpyrrolidin-2-yl, 1-ethylpyrrolidin-2-yl, 1-propylpyrrolidin-2-yl, 1-isopropylpyrrolidin-2-yl, pyrrolidin-1-yl, piperidin-1-yl, 1-methylpiperazin-4-yl, 1-ethylpiperazin-4-yl, morpholinyl, tetrahydrofuran-2-yl, tetrahydrofuran-3-yl, tetrahydropyran-2-yl, tetrahydropyran-3-yl, tetrahydropyran-4-yl, thiomorpholinyl, dimethylamino, diethylamino, dipropylamino, diisopropylamino, methylethylamino, methylpropylamino, methylamino, ethylamino, propylamino, isopropylamino, cyclopropylamino, cyclobutylamino, methylisopropylamino, N-methyl-N-cyclopropylamino, N-methyl-N-cyclobutylamino or ethylpropylamino.

4. The compound according to claim 1, a stereoisomer thereof, and a pharmaceutically acceptable salt thereof, wherein R₂ is C₁-C₄ alkyl, which is unsubstituted or substituted with 1 to 3 substituents selected from fluorine, chlorine, cyano, methyl, ethyl, propyl, isopropyl, methoxy, ethoxy, propoxy, isopropoxy, methylthio, ethylthio, propylthio, isopropylthio, hydroxyl, cyclopropyl, cyclobutyl, methylsulfonyl, ethylsulfonyl, propylsulfonyl and isopropylsulfonyl.

5. The compound according to claim 4, a stereoisomer thereof, and a pharmaceutically acceptable salt thereof, wherein R₂ is methyl, ethyl, propyl, isopropyl, hydroxyethyl, hydroxypropyl, trifluoromethyl, fluoroethyl, fluoropropyl, 2,2,2-trifluoroethyl, 2,2-difluoroethyl, 3,3,3-trifluoropropyl, methoxyethyl, methoxypropyl, ethoxyethyl, ethoxypropyl, methylthioethyl, methylthiopropyl, ethylthioethyl, ethylthiopropyl, 2-hydroxy-2-methylpropyl, 3-hydroxy-3-methylbutyl, methylsulfonylpropyl, methylsulfonylethyl, ethylsulfonylethyl, ethylsulfonylpropyl, isopropylsulfonylethyl, or isopropylsulfonylpropyl.

6. The compound according to claim 1, a stereoisomer thereof, and a pharmaceutically acceptable salt thereof, wherein R₃, R₄ and R₅ are each independently hydrogen, fluorine, chlorine, or bromine, and at least one of R₃, R₄, and R₅ is fluorine, chlorine, or bromine.

7. The compound according to claim 6, a stereoisomer thereof, and a pharmaceutically acceptable salt thereof, wherein R₃ and R₅ are each independently hydrogen, fluorine, or chlorine, and R₄ is chlorine.

8. The compound according to claim 1, a stereoisomer thereof, and a pharmaceutically acceptable salt thereof, wherein the compound is selected from:

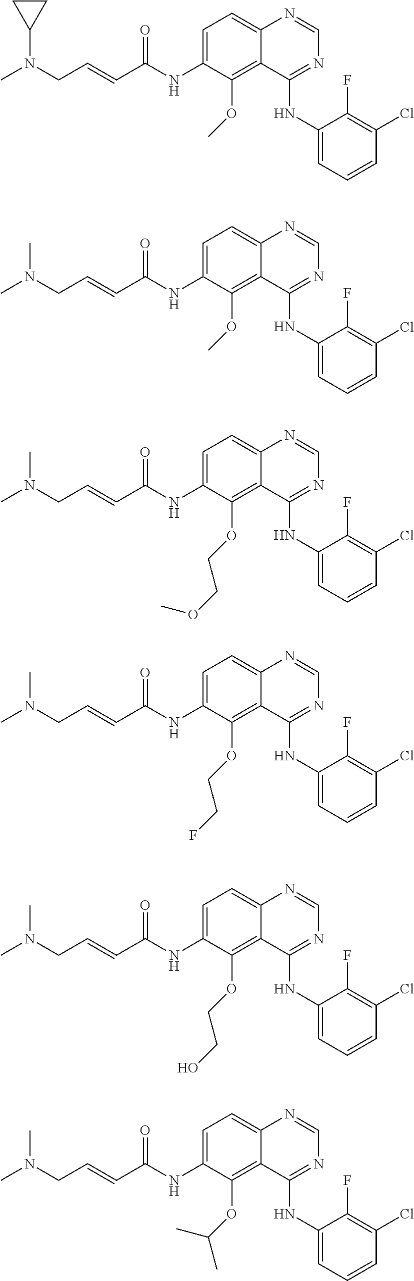 [SMILES: COc1c(NC(=O)/C=C/CN(C)C2CC2)ccc2ncnc(Nc3cccc(Cl)c3F)c12 ; COc1c(NC(=O)/C=C/CN(C)C)ccc2ncnc(Nc3cccc(Cl)c3F)c12 ; COCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCF ; CN(C)C/C=C/C(=O)Nc1ccc2ncnc(Nc3cccc(Cl)c3F)c2c1OCCO ; CC(C)Oc1c(NC(=O)/C=C/CN(C)C)ccc2ncnc(Nc3cccc(Cl)c3F)c12]

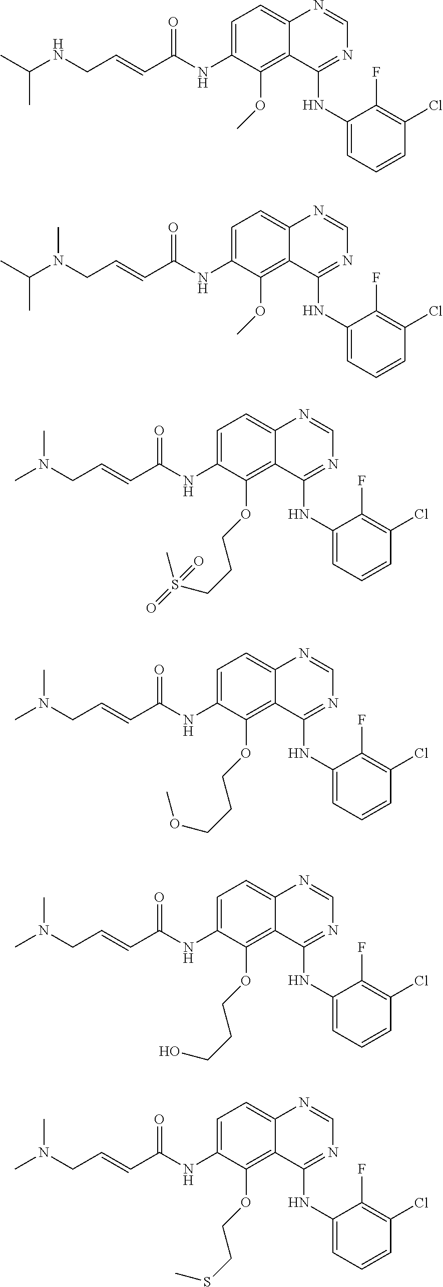 [SMILES: COc1c(NC(=O)/C=C/CNC(C)C)ccc2ncnc(Nc3cccc(Cl)c3F)c12 ; COc1c(NC(=O)/C=C/CN(C)C(C)C)ccc2ncnc(Nc3cccc(Cl)c3F)c12 ; CN(C)C/C=C/C(=O)Nc1ccc2ncnc(Nc3cccc(Cl)c3F)c2c1OCCCS(C)(=O)=O ; COCCCOc1c(NC(=O)/C=C/CN(C)C)ccc2ncnc(Nc3cccc(Cl)c3F)c12 ; CN(C)C/C=C/C(=O)Nc1ccc2ncnc(Nc3cccc(Cl)c3F)c2c1OCCCO ; CSCCOc1c(NC(=O)/C=C/CN(C)C)ccc2ncnc(Nc3cccc(Cl)c3F)c12]

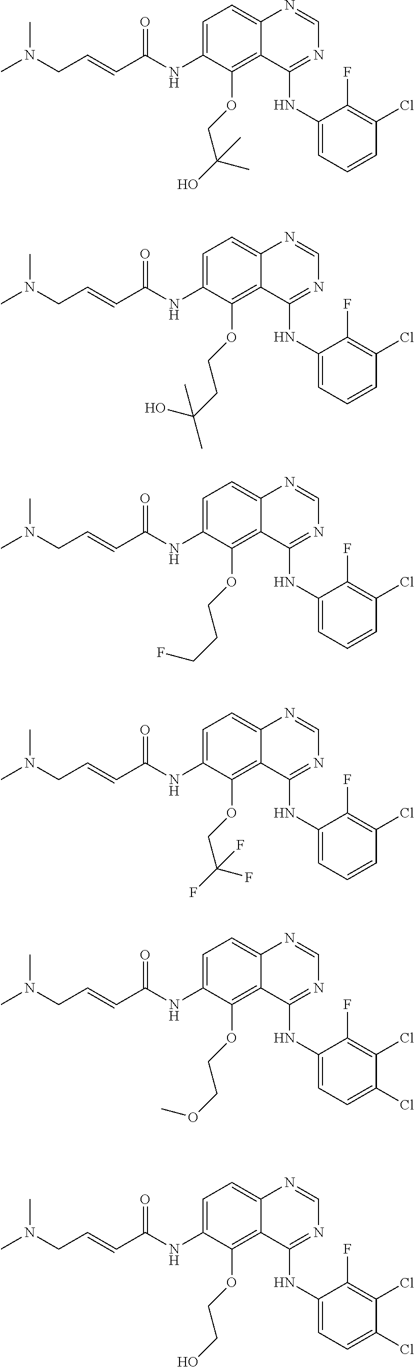

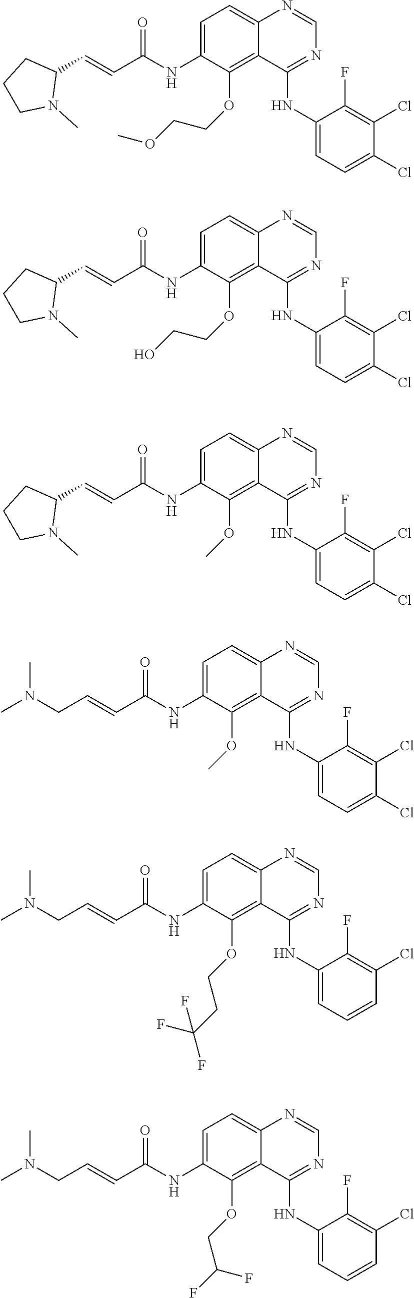

9. A pharmaceutical composition comprising the compound of claim 1, a stereoisomer thereof, or a pharmaceutically acceptable salt thereof, and one or more pharmaceutically acceptable carriers or excipients.

10. The pharmaceutical composition according to claim 9, wherein the pharmaceutical composition also contains one or more other therapeutic agents.

11. A method of treating tyrosine kinase EGFR- or HER2-related cancers and autoimmune diseases in a subject in need thereof, comprising administering to the subject the compound according to claim 1, or a pharmaceutically acceptable salt, isomer, solvate, or prodrug thereof, wherein the cancer and autoimmune diseases include: fundus diseases, dry eye, psoriasis, vitiligo, dermatitis, alopecia areata, rheumatoid arthritis, colitis, multiple sclerosis, systemic lupus erythematosus, Crohn's disease, atherosclerosis, pulmonary fibrosis, liver fibrosis, myelofibrosis, non-small cell lung cancer, small cell lung cancer, breast cancer, pancreatic cancer, glioma, glioblastoma, ovarian cancer, cervical cancer, colorectal cancer, melanoma, endometrial cancer, prostate cancer, bladder cancer, leukemia, stomach cancer, liver cancer, gastrointestinal stromal tumor, thyroid cancer, chronic myelogenous leukemia, acute myeloid leukemia, non-Hodgkin lymphoma, nasopharyngeal cancer, esophageal cancer, brain tumors, B-cell and T-cell lymphoma, lymphoma, multiple myeloma, biliary carcinosarcoma, and cholangiocarcinoma.

12. The compound according to claim 6, a stereoisomer thereof, and a pharmaceutically acceptable salt thereof, wherein R₃ is hydrogen, fluorine, or chlorine, R₄ is chlorine, and R₅ is fluorine.

13. A method of treating diseases related to brain metastasis in a subject in need thereof, comprising administering to the subject the compound according to claim 1, or a pharmaceutically acceptable salt, isomer, solvate, or prodrug thereof.

14. A method of treating tyrosine kinase EGFR- or HER2-related cancers and autoimmune diseases in a subject in need thereof, comprising administering to the subject the pharmaceutical composition according to claim 9, wherein the cancer and autoimmune diseases include: fundus diseases, dry eye, psoriasis, vitiligo, dermatitis, alopecia areata, rheumatoid arthritis, colitis, multiple sclerosis, systemic lupus erythematosus, Crohn's disease, atherosclerosis, pulmonary fibrosis, liver fibrosis, myelofibrosis, non-small cell lung cancer, small cell lung cancer, breast cancer, pancreatic cancer, glioma, glioblastoma, ovarian cancer, cervical cancer, colorectal cancer, melanoma, endometrial cancer, prostate cancer, bladder cancer, leukemia, stomach cancer, liver cancer, gastrointestinal stromal tumor, thyroid cancer, chronic myelogenous leukemia, acute myeloid leukemia, non-Hodgkin lymphoma, nasopharyngeal cancer, esophageal cancer, brain tumors, B-cell and T-cell lymphoma, lymphoma, multiple myeloma, biliary carcinosarcoma, and cholangiocarcinoma.

15. A method of treating diseases related to brain metastasis in a subject in need thereof, comprising administering to the subject the pharmaceutical composition according to claim 9.
